(19) United States
(12) Patent Application Publication
(10) Pub. No.: US 2022/0241253 A1
(43) Pub. Date: Aug. 4, 2022

(54) USE OF GABOXADOL IN THE TREATMENT OF SUBSTANCE USE DISORDERS

(71) Applicant: Ovid Therapeutics, Inc., New York, NY (US)

(72) Inventor: Matthew During, Weston, CT (US)

(21) Appl. No.: 17/723,979

(22) Filed: Apr. 19, 2022

Related U.S. Application Data
(63) Continuation of application No. 16/377,172, filed on Apr. 6, 2019.
(60) Provisional application No. 62/653,641, filed on Apr. 6, 2018.
(60) Provisional application No. 62/654,786, filed on Apr. 9, 2018.

(51) Int. Cl. A61K 31/437; A61P 25/32; A61K 45/06; A61P 25/36; A61P 25/34

(52) U.S. Cl. CPC A61K 31/437; A61P 25/32; A61P 25/34; A61P 25/36; A61K 45/06

(57) ABSTRACT

Methods of using gaboxadol or a pharmaceutically acceptable salt thereof and compositions of gaboxadol or a pharmaceutically acceptable salt thereof for use in treating a substance use disorder, preventing a substance use craving, diminishing a substance use craving, and/or facilitating substance use cessation are provided. Substance use disorders include alcohol use disorder, caffeine use disorder, cannabis use disorder, hallucinogen use disorder, inhalant use disorder, opioid use disorder, sedative use disorder, stimulant use disorder, tobacco use disorder and nicotine use disorder.

### CROSS-REFERENCE TO RELATED APPLICATION

This application is a continuation application of U.S. patent application Ser. No. 16/377,172, filed Apr. 6, 2019, which claims benefit and priority to U.S. Provisional Application No. 62/653,641, filed Apr. 6, 2018 and U.S. Provisional Application No. 62/654,786, filed Apr. 9, 2018, which are incorporated herein by reference in their entireties.

### TECHNICAL FIELD

Methods of using gaboxadol or a pharmaceutically acceptable salt thereof and pharmaceutical compositions containing gaboxadol or a pharmaceutically acceptable salt thereof useful for treating substance use disorders are provided.

### BACKGROUND

According to the NIH National Institute on Drug Abuse (NIDA), abuse of and addiction to alcohol, nicotine, and illicit and prescription drugs cost Americans more than $700 billion a year in increased health care costs, crime, and lost productivity. Addiction may be defined as a chronic, relapsing brain disease that is characterized by compulsive drug seeking and use, despite harmful consequences. It is considered a brain disease because drugs change the brain—they change its structure and how it works. These brain changes can be long-lasting, and can lead to the harmful behaviors seen in people who abuse drugs. According to the NIDA, the term addiction may be regarded as equivalent to a severe substance use disorder as defined by the Diagnostic and Statistical Manual of Mental Disorders, Fifth Edition (DSM-5, 2013).

Substance use disorders occur when the recurrent use of alcohol and/or drugs causes clinically and functionally significant impairment, such as health problems, disability, and failure to meet major responsibilities at work, school, or home. According to the DSM-5, a diagnosis of substance use disorder is based on evidence of impaired control, social impairment, risky use, and pharmacological criteria. The DSM-5 establishes nine types of Substance-Related Disorders: 1. Alcohol, 2. Caffeine (substance use disorder does not apply to caffeine), 3. Cannabis (e.g., marijuana), 4. Hallucinogens, 5. Inhalants, 6. Opioids (e.g., heroin), 7. Sedatives, Hypnotics, or Anxiolytics (e.g., benzodiazepines, barbiturates), 8. Stimulants (e.g., cocaine, methamphetamine), and 9. Tobacco.

According to the DSM 5, each specific substance (other than caffeine) is addressed as a separate use disorder (e.g., alcohol use disorder, stimulant use disorder, etc.), but nearly all substances are diagnosed based on the same overarching criteria. A list of eleven symptoms of substance use disorders is provided in the DSM-5 and set forth below. Severity of the disorder is based on the number of symptoms exhibited: mild substance use disorder requires two to three symptoms, four or five symptoms indicate moderate substance use disorder, and greater than six symptoms indicates severe substance use disorder.
1. substance is often taken in larger amounts or over a longer period of time than was intended
2. persistent desire or unsuccessful efforts to cut down or control substance use
3. great deal of time is spent in activities necessary to obtain the substance, use the substance, or recover from its effects
4. craving or strong desire to use the substance
5. recurrent use resulting in failure to fulfill major role obligations at work, school, home
6. continued substance use despite having persistent or recurrent social or interpersonal problems
7. important social, occupational, or recreational activities are given up or reduced because of substance use
8. recurrent substance use in situations in which it is physically hazardous
9. substance use is continued despite knowledge of having a persistent or recurrent physical or psychological problem that is likely to have been caused or exacerbated by the substance
10. tolerance, as defined by either of the following:
   a. a need for markedly increased amounts of the substance to achieve intoxication or desired effect
   b. a markedly diminished effect with continued use of the same amount of substance
11. withdrawal, as manifested by either of the following:
   a. characteristic withdrawal syndrome for the substance
   b. use of the substance or closely related substance is taken to relieve or avoid withdrawal symptoms

Tobacco addiction represents a leading cause of preventable illness and premature death in our society. According to the 2014 U.S Department of Health and Human Services, The Health Consequences of Smoking—50 Years of Progress: Report of the Surgeon General, more than 20 million Americans have died as a result of smoking tobacco since the first Surgeon General's report on smoking and health was released in 1964. Active smoking of tobacco affects nearly every organ of the body. It has been causally associated with the following cancers: oropharynx, larynx, esophagus, trachea, bronchus and lung, acute myeloid leukemia, stomach, pancreas, kidney and ureter, cervix and bladder. Smoking has been causally associated with the following chronic diseases: stroke, blindness, cataracts, periodontitis, aortic aneurysm, early abdominal aortic atherosclerosis in young adults, coronary heart disease, pneumonia, atherosclerotic peripheral vascular disease, chronic obstructive pulmonary disease, asthma, reduced fertility in women, hip fractures, and overall diminished health. In addition, active smoking is now causally associated with age-related macular degeneration, diabetes, colorectal cancer, liver cancer, adverse health outcomes in cancer patients and survivors, tuberculosis, erectile dysfunction, orofacial clefts in infants, ectopic pregnancy, rheumatoid arthritis, inflammation, and impaired immune function.

According to NIDA, most smokers use tobacco regularly because they are addicted to nicotine. Addiction is characterized by compulsive drug-seeking and use, even in the face of negative health consequences. Cigarette smoking is the most popular method of using tobacco; however, many people also use smokeless tobacco products, such as snuff and chewing tobacco, which also contain nicotine. Administration of nicotine causes a transient surge of endorphins in the reward circuits of the brain which results in a slight, brief euphoria. However, like other drugs of abuse, nicotine increases levels of the neurotransmitter dopamine in these reward circuits which reinforces the behavior of taking the drug. For many tobacco users, the long-term brain changes induced by continued nicotine exposure result in addiction, which involves withdrawal symptoms when not smoking. Being without nicotine for too long can cause a regular user to experience irritability, craving, depression, anxiety, cognitive and attention deficits, sleep disturbances, and increased appetite. The majority of smokers would like to stop smoking, and each year about half try to quit permanently. Yet, only about 6 percent of smokers are able to quit in a given year.

Substance abuse and addiction are public health issues with significant social and economic impact on both the addict and society. There is a need in the art for new methods for treating and preventing substance use disorders and the relapse use of addictive agents. For example, there is a need to help tobacco users quit using tobacco and nicotine.

Gaboxadol (4,5,6,7-tetrahydroisoxazolo [5,4-c]pyridine-3-ol) (THIP)) is described in EP Patent No. 0000338 and in EP Patent No. 0840601, U.S. Pat. Nos. 4,278,676, 4,362,731, 4,353,910, and WO 2005/094820. Gaboxadol is a selective GABAA receptor agonist with a preference for 6-subunit containing GABAA receptors. In the early 1980s gaboxadol was the subject of a series of pilot studies that tested its efficacy as an analgesic and anxiolytic, as well as a treatment for tardive dyskinesia, Huntington's disease, Alzheimer's disease, and spasticity. In the 1990s gaboxadol moved into late stage development for the treatment of insomnia. The development was discontinued after the compound failed to show significant effects in sleep onset and sleep maintenance in a three-month efficacy study. Additionally, patients with a history of drug abuse who received gaboxadol experienced a steep increase in psychiatric adverse events.

### SUMMARY

A method of treating a substance use disorder is provided which includes administering to a patient in need thereof a pharmaceutical composition including gaboxadol or a pharmaceutically acceptable salt thereof. A method of treating a substance use disorder is provided which includes administering to a patient in need thereof an effective amount of gaboxadol or a pharmaceutically acceptable salt thereof. A method of treating a substance use disorder is provided which includes administering to a patient in need thereof an amount of gaboxadol or a pharmaceutically acceptable salt thereof which is effective to reduce one or more symptoms of the substance use disorder. Eleven symptoms of substance use disorders are provided above. A method for preventing a substance use craving in a patient in need thereof is provided which includes administering to the patient a prophylactically effective amount of gaboxadol or a pharmaceutically acceptable salt thereof. A method for diminishing a substance use craving in a patient in need thereof is provided which includes administering to the patient an effective amount of gaboxadol or a pharmaceutically acceptable salt thereof. Also provided herein are pharmaceutical compositions for use in treating a substance use disorder.

A method of treating nicotine use disorder is provided which includes administering to a patient in need thereof a pharmaceutical composition including gaboxadol or a pharmaceutically acceptable salt thereof. A method of treating nicotine use disorder is provided which includes administering to a patient in need thereof an effective amount of gaboxadol or a pharmaceutically acceptable salt thereof. A method of treating nicotine use disorder is provided which includes administering to a patient in need thereof an amount of gaboxadol or a pharmaceutically acceptable salt thereof which is effective to reduce one or more symptoms of the nicotine use disorder. A method for preventing a nicotine craving in a patient in need thereof is provided which includes administering to the patient a prophylactically effective amount of gaboxadol or a pharmaceutically acceptable salt thereof. A method for diminishing a nicotine craving in a patient in need thereof is provided which includes administering to the patient an effective amount of gaboxadol or a pharmaceutically acceptable salt thereof. Also provided herein are pharmaceutical compositions for use in treating nicotine use disorder.

A method of treating tobacco use disorder is provided which includes administering to a patient in need thereof a pharmaceutical composition including gaboxadol or a pharmaceutically acceptable salt thereof. A method of treating tobacco use disorder is provided which includes administering to a patient in need thereof an effective amount of gaboxadol or a pharmaceutically acceptable salt thereof. A method of treating tobacco use disorder is provided which includes administering to a patient in need thereof an amount of gaboxadol or a pharmaceutically acceptable salt thereof which is effective to reduce one or more symptoms of the tobacco use disorder. A method for preventing a tobacco craving in a patient in need thereof is provided which includes administering to the patient a prophylactically effective amount of gaboxadol or a pharmaceutically acceptable salt thereof. A method for diminishing a tobacco craving in a patient in need thereof is provided which includes administering to the patient an effective amount of gaboxadol or a pharmaceutically acceptable salt thereof. Also provided herein are pharmaceutical compositions for use in treating tobacco use disorder.

A method of facilitating smoking cessation is provided which includes administering to a patient in need thereof a pharmaceutical composition including gaboxadol or a pharmaceutically acceptable salt thereof.

### DETAILED DESCRIPTION

Methods of using gaboxadol or a pharmaceutically acceptable salt thereof and compositions of gaboxadol or a pharmaceutically acceptable salt thereof for use in treating a substance use disorder, preventing a substance use craving, diminishing a substance use craving, and/or facilitating substance use cessation are provided. Substance use disorders involve abuse of psychoactive compounds such as alcohol, caffeine, cannabis, hallucinogens, inhalants, opioids, sedatives, hypnotics, anxiolytics, stimulants, and tobacco. As used herein “substance” or “substances” are psychoactive compounds which can be addictive such as alcohol, caffeine, cannabis, hallucinogens, inhalants, opioids, sedatives, hypnotics, anxiolytics, stimulants, nicotine and tobacco. In embodiments, methods of using gaboxadol or a pharmaceutically acceptable salt thereof and compositions of gaboxadol or a pharmaceutically acceptable salt thereof for use in treating nicotine use disorder, preventing nicotine craving, diminishing nicotine craving, and/or facilitating nicotine use cessation are provided. In embodiments, methods of using gaboxadol or a pharmaceutically acceptable salt thereof and compositions of gaboxadol or a pharmaceutically acceptable salt thereof for use in treating tobacco use disorder, preventing tobacco craving, diminishing tobacco craving, and/or facilitating tobacco use cessation are provided. In embodiments, methods of using gaboxadol or a pharmaceutically acceptable salt thereof and compositions of gaboxadol or a pharmaceutically acceptable salt thereof for use in facilitating smoking cessation are provided.

In embodiments, methods of using gaboxadol or a pharmaceutically acceptable salt thereof and compositions of gaboxadol or a pharmaceutically acceptable salt thereof for use in treating alcohol use disorder, preventing alcohol craving, diminishing alcohol craving, and/or facilitating alcohol use cessation are provided. In embodiments, methods of using gaboxadol or a pharmaceutically acceptable salt thereof and compositions of gaboxadol or a pharmaceutically acceptable salt thereof for use in for treating caffeine use disorder, preventing caffeine craving, diminishing caffeine craving, and/or facilitating caffeine use cessation are provided. In embodiments, methods of using gaboxadol or a pharmaceutically acceptable salt thereof and compositions of gaboxadol or a pharmaceutically acceptable salt thereof for use in treating cannabis use disorder, preventing cannabis craving, diminishing cannabis craving, and/or facilitating cannabis use cessation are provided. In embodiments, methods of using gaboxadol or a pharmaceutically acceptable salt thereof and compositions of gaboxadol or a pharmaceutically acceptable salt thereof for use in treating hallucinogen use disorder, preventing hallucinogen craving, diminishing hallucinogen craving, and/or facilitating hallucinogen use cessation are provided. In embodiments, methods of using gaboxadol or a pharmaceutically acceptable salt thereof and compositions of gaboxadol or a pharmaceutically acceptable salt thereof for use in treating inhalant use disorder, preventing inhalant craving, diminishing inhalant craving, and/or facilitating inhalant use cessation are provided. In embodiments, methods of using gaboxadol or a pharmaceutically acceptable salt thereof and compositions of gaboxadol or a pharmaceutically acceptable salt thereof for use in treating opioid use disorder, preventing opioid craving, diminishing opioid craving, and/or facilitating opioid use cessation are provided. In embodiments, methods of using gaboxadol or a pharmaceutically acceptable salt thereof and compositions of gaboxadol or a pharmaceutically acceptable salt thereof for use in treating sedative use disorder, preventing sedative craving, diminishing sedative craving, and/or facilitating sedative use cessation are provided. In embodiments, methods of using gaboxadol or a pharmaceutically acceptable salt thereof and compositions of gaboxadol or a pharmaceutically acceptable salt thereof for use in treating stimulant use disorder, preventing stimulant craving, diminishing stimulant craving, and/or facilitating stimulant use cessation are provided.

As used herein, cannabis includes materials which include delta-9-tetrahydrocannabinol (THC) such as marijuana, hashish, and THC extracts such as hash oil, wax, butter, tincture and the like. Hallucinogens include LSD, DMT, PCP, DMT, MDMA, psilocybin, mescaline, and ketamine. Inhalants are volatile substances that produce chemical vapors that can be inhaled to produce a psychoactive effect. Inhalants include volatile solvents such as toluene, ether, and gasoline, which can be found, e.g., in paint thinners and removers, aerosol cans, dry-cleaning fluids, degreasers, glues, correction fluids, and felt-tip markers. Inhalants also include gases such as nitrous oxide and butane. Opioids include opium, heroin, morphine, codeine, oxycodone, hydrocodone, hydromorphone, oxymorphone, fentanyl, methadone, meperidine, tramadol, carfentanil and buprenorphine. Anxiolytics, sedatives and hypnotics (referred to collectively herein as sedatives) include benzodiazepines such as diazepam, alprazolam, clobazam, clonazepam, trazolam, temazepam and lorazepam, barbiturates such as secobarbital, amobarbital, butabarbital, thiopental, and others such as chloral hydrate, hydroxyzine, promethazine, eszopiclone, zaleplon, zolpidem, zopiclone, and diphenhydramine. Stimulants include methylphenidate, dexmethylphenidate, cocaine, 3,4-methylenedioxymethamphetamine (MDMA), atomexetine, lisdexamfetamine, caffeine, phenylephrine, pseudoephedrine, and amphetamines such as methamphetamine and dextroamphetamine.

In embodiments, the subject is first determined or diagnosed to have a substance use disorder, or to be at risk of developing a substance use disorder, by diagnostic testing, observation or analysis by a medical care provider using, for example, the eleven DSM 5 criteria. An effective amount of gaboxadol or a pharmaceutically acceptable salt thereof, or an effective amount of gaboxadol or a pharmaceutically acceptable salt thereof and one or more additional therapeutic agents, are then provided to the subject for treatment or prevention of the substance use disorder. As used herein, an “effective amount” or “therapeutically effective amount” is a dosage sufficient to treat, prevent, inhibit, or alleviate one or more symptoms of the substance use disorder, e.g., the eleven symptoms listed in the DSM 5. For example, in the context of treating or preventing a substance use disorder using the methods described herein, an effective amount of gaboxadol or a pharmaceutically acceptable salt thereof can be the amount sufficient to cause the subject to reduce or discontinue use of a substance. The precise dosage of gaboxadol or a pharmaceutically acceptable salt thereof will vary according to a variety of factors such as subject-dependent variables (e.g., age, immune system health, clinical symptoms etc.). The dosage of the gaboxadol or a pharmaceutically acceptable salt thereof, or the gaboxadol or a pharmaceutically acceptable salt thereof and the one or more additional therapeutic agents, may be specifically determined by the medical practitioner for treatment or prevention of the substance use disorder.

In embodiments, a subject may be considered at risk of a substance use disorder or relapse to use a substance when the subject has previously been addicted to the same or a different addictive substance. In embodiments, the subject is considered at risk of a substance use disorder or relapse to use a substance when the subject is psychologically addicted to a substance, even if the subject is no longer physically addicted.

In embodiments, the subject is addicted to or is at risk of becoming addicted to a therapeutic substance provided to the subject to treat a disease or disorder, e.g., a pain medication. In embodiments, the subject may be at risk of abusing an addictive therapeutic substance, such as a pain medication. Abusing an addictive therapeutic substance, in embodiments, may be understood to indicate using the substance for a reason different than or in addition to its prescribed use. In such a situation, a subject may be provided with both an addictive therapeutic substance and gaboxadol or a pharmaceutically acceptable salt thereof, alone or in combination with an additional therapeutic agent. For example, a subject suffering from pain, or at risk of pain, may be provided with an opioid agonist and gaboxadol or a pharmaceutically acceptable salt thereof, to both provide analgesia and prevent or treat addiction to the opioid agonist.

Relapse use refers to the process of returning to the use of a substance after a period of abstinence from, or limited or reduced use of, the substance. In certain situations, relapse use of a substance refers to the return to use of a substance by a subject who has undergone physical withdrawal from the a substance. Typically, the subject will have undergone physical withdrawal from the substance during a period of non-use or limited or reduced use of the substance. In embodiments, relapse use can occur in a subject who has stopped using a substance without using any anti-addiction agents. In embodiments, relapse use can occur in a subject who has previously undergone a treatment regime with an effective amount of an anti-addiction agent to reduce or eliminate use of a substance, but who is no longer using an effective amount of the anti-addiction agent. Anti-addictive agents include any and all agents used to treat or prevent addiction or withdrawal symptoms. It is believed that molecular mechanisms underlying relapse are common to different classes of drugs of abuse. Drug craving and loss of control over drug taking behavior associated with relapse can be under the direct influence of stress and environmental conditioning stimuli; two major factors affecting resumption to drug use.

In embodiments, the substance use disorder is nicotine use disorder. Accordingly, the subject may be any patient who uses nicotine in any form, including cigarettes, electronic cigarettes or vaporizers (“vaping”), chewing tobacco, cigars, snuff, pipes, hookahs, transdermal patches, gum, lozenges, and the like. In embodiments, the patient is physically addicted to nicotine. In embodiments, the patient is psychologically addicted to nicotine. In embodiments, the patient is addicted to tobacco. In embodiments, the patient is physically addicted to tobacco. In embodiments, the patient is psychologically addicted to tobacco.

In embodiments, methods and compositions for treating nicotine use disorder, preventing nicotine craving, diminishing nicotine craving, and/or facilitating nicotine use cessation in a subject, include administering to the patient in need thereof an effective amount of gaboxadol or a pharmaceutically acceptable salt thereof. In embodiments, methods and compositions for treating nicotine use disorder, preventing nicotine craving, diminishing nicotine craving, and/or facilitating nicotine use cessation in a subject, include administering to the patient in need thereof a prophylactically effective amount of gaboxadol or a pharmaceutically acceptable salt thereof.

In embodiments, methods and compositions for treating tobacco use disorder, preventing tobacco craving, diminishing tobacco craving, and/or facilitating tobacco use cessation in a subject, include administering to the patient in need thereof an effective amount of gaboxadol or a pharmaceutically acceptable salt thereof. In embodiments, methods and compositions for treating tobacco use disorder, preventing tobacco craving, diminishing tobacco craving, and/or facilitating tobacco use cessation in a subject, include administering to the patient in need thereof a prophylactically effective amount of gaboxadol or a pharmaceutically acceptable salt thereof.

In embodiments, methods of using gaboxadol or a pharmaceutically acceptable salt thereof and compositions of gaboxadol or a pharmaceutically acceptable salt thereof for reducing the rate of maternal postpartum smoking relapse are provided. In embodiments, methods of preventing and/or reducing postpartum smoking relapse in mothers include administering gaboxadol or a pharmaceutically acceptable salt thereof. In embodiments, methods of increasing postpartum maternal smoking abstinence using gaboxadol or a pharmaceutically acceptable salt thereof are provided. Postpartum maternal smoking abstinence and/or postpartum smoking relapse may be defined as maintaining smoke-free status continuously for a specified period of time. For example, smoke-free status may be measure at follow-up visits post partum, e.g., after 1 month 2-months 3-months, 6 months etc. Abstinence may be biochemically verified. For example, biologic verification may be determined through expired carbon monoxide (CO) at, e.g., 6-months postpartum. Additional measures may include maternal number of cigarettes smoked; maternal self-efficacy scores on the Romanian version of the General Self Efficacy scale; maternal motivation scores on the Reasons for Quitting scale; and/or self-reported smoke status of the husband/partner.

In embodiments, alcohol is the subject of the substance use disorder. In embodiments, the patient is addicted to alcohol. In embodiments, the patient is physically addicted to alcohol. In embodiments, the patient is psychologically addicted to alcohol. In embodiments, methods and compositions for treating alcohol use disorder, preventing alcohol craving, diminishing alcohol craving, and/or facilitating alcohol use cessation in a subject, include administering to the patient in need thereof an effective amount of gaboxadol or a pharmaceutically acceptable salt thereof. In embodiments, methods and compositions for treating alcohol use disorder, preventing alcohol craving, diminishing alcohol craving, and/or facilitating alcohol use cessation in a subject, include administering to the patient in need thereof a prophylactically effective amount of gaboxadol or a pharmaceutically acceptable salt thereof.

In embodiments, caffeine is the subject of the substance use disorder. In embodiments, the patient is addicted to caffeine. In embodiments, the patient is physically addicted to caffeine. In embodiments, the patient is psychologically addicted to caffeine. In embodiments, methods and compositions for treating caffeine use disorder, preventing caffeine craving, diminishing caffeine craving, and/or facilitating caffeine use cessation in a subject, include administering to the patient in need thereof an effective amount of gaboxadol or a pharmaceutically acceptable salt thereof. In embodiments, methods and compositions for treating caffeine use disorder, preventing caffeine craving, diminishing caffeine craving, and/or facilitating caffeine use cessation in a subject, include administering to the patient in need thereof a prophylactically effective amount of gaboxadol or a pharmaceutically acceptable salt thereof.

In embodiments, cannabis is the subject of the substance use disorder. In embodiments, the patient is addicted to cannabis. In embodiments, the patient is physically addicted to cannabis. In embodiments, the patient is psychologically addicted to cannabis. In embodiments, methods and compositions for treating cannabis use disorder, preventing cannabis craving, diminishing cannabis craving, and/or facilitating cannabis use cessation in a subject, include administering to the patient in need thereof an effective amount of gaboxadol or a pharmaceutically acceptable salt thereof. In embodiments, methods and compositions for treating cannabis use disorder, preventing cannabis craving, diminishing cannabis craving, and/or facilitating cannabis use cessation in a subject, include administering to the patient in need thereof a prophylactically effective amount of gaboxadol or a pharmaceutically acceptable salt thereof.

In embodiments, a hallucinogen is the subject of the substance use disorder. In embodiments, the patient is addicted to a hallucinogen. In embodiments, the patient is physically addicted to a hallucinogen. In embodiments, the patient is psychologically addicted to a hallucinogen. In embodiments, methods and compositions for treating hallucinogen use disorder, preventing hallucinogen craving, diminishing hallucinogen craving, and/or facilitating hallucinogen use cessation in a subject, include administering to the patient in need thereof an effective amount of gaboxadol or a pharmaceutically acceptable salt thereof. In embodiments, methods and compositions for treating hallucinogen use disorder, preventing hallucinogen craving, diminishing hallucinogen craving, and/or facilitating hallucinogen use cessation in a subject, include administering to the patient in need thereof a prophylactically effective amount of gaboxadol or a pharmaceutically acceptable salt thereof.

In embodiments, an inhalant is the subject of the substance use disorder. In embodiments, the patient is addicted to inhalants. In embodiments, the patient is physically addicted to inhalants. In embodiments, the patient is psychologically addicted to inhalants. In embodiments, methods and compositions for treating inhalant use disorder, preventing inhalant craving, diminishing inhalant craving, and/or facilitating inhalant use cessation in a subject, include administering to the patient in need thereof an effective amount of gaboxadol or a pharmaceutically acceptable salt thereof. In embodiments, methods and compositions for treating inhalant use disorder, preventing inhalant craving, diminishing inhalant craving, and/or facilitating inhalant use cessation in a subject, include administering to the patient in need thereof a prophylactically effective amount of gaboxadol or a pharmaceutically acceptable salt thereof.

In embodiments, opioids are the subject of the substance use disorder. In embodiments, the patient is addicted to an opioid. In embodiments, the patient is physically addicted to an opioid. In embodiments, the patient is psychologically addicted to an opioid. In embodiments, methods and compositions for treating opioid use disorder, preventing opioid craving, diminishing opioid craving, and/or facilitating opioid use cessation in a subject, include administering to the patient in need thereof an effective amount of gaboxadol or a pharmaceutically acceptable salt thereof. In embodiments, methods and compositions for treating opioid use disorder, preventing opioid craving, diminishing opioid craving, and/or facilitating opioid use cessation in a subject, include administering to the patient in need thereof a prophylactically effective amount of gaboxadol or a pharmaceutically acceptable salt thereof.

In embodiments, sedatives are the subject of the substance use disorder. In embodiments, the patient is addicted to a sedative. In embodiments, the patient is physically addicted to a sedative. In embodiments, the patient is psychologically addicted to a sedative. In embodiments, methods and compositions for treating sedative use disorder, preventing sedative craving, diminishing sedative craving, and/or facilitating sedative use cessation in a subject, include administering to the patient in need thereof an effective amount of gaboxadol or a pharmaceutically acceptable salt thereof. In embodiments, methods and compositions for treating sedative use disorder, preventing sedative craving, diminishing sedative craving, and/or facilitating sedative use cessation in a subject, include administering to the patient in need thereof a prophylactically effective amount of gaboxadol or a pharmaceutically acceptable salt thereof.

In embodiments, stimulants are the subject of the substance use disorder. In embodiments, the patient is addicted to a stimulant. In embodiments, the patient is physically addicted to a stimulant. In embodiments, the patient is psychologically addicted to a stimulant. In embodiments, methods and compositions for treating stimulant use disorder, preventing stimulant craving, diminishing stimulant craving, and/or facilitating stimulant use cessation in a subject, include administering to the patient in need thereof an effective amount of gaboxadol or a pharmaceutically acceptable salt thereof. In embodiments, methods and compositions for treating stimulant use disorder, preventing stimulant craving, diminishing stimulant craving, and/or facilitating stimulant use cessation in a subject, include administering to the patient in need thereof a prophylactically effective amount of gaboxadol or a pharmaceutically acceptable salt thereof.

Many pharmaceutical products are administered as a fixed dose, at regular intervals, to achieve therapeutic efficacy. The duration of action is reflected by its plasma half-life. Since efficacy is often dependent on sufficient exposure within the central nervous system administration of CNS drugs with a short half-life may require frequent maintenance dosing. Advantageously disclosed herein are methods for treating a substance use disorder, preventing a substance use craving, diminishing a substance use craving, and/or facilitating substance use cessation by administration of gaboxadol or pharmaceutically acceptable salt thereof. For example, in embodiments, methods for treating a substance use disorder, preventing a substance use craving, diminishing a substance use craving, and/or facilitating substance use cessation are provided which include administering to a patient in need thereof a pharmaceutical composition including about 0.05 mg to about 75 mg of gaboxadol or pharmaceutically acceptable salt thereof. In embodiments, the composition provides improvement for more than 6 hours after administration to the patient.

For example, dosages may include amounts of an gaboxadol or pharmaceutically acceptable salt thereof in the range of about, e.g., 1 mg to 30 mg, 1 mg to 20 mg, 1 mg to 15 mg, 0.01 mg to 10 mg, 0.1 mg to 15 mg, 0.15 mg to 12.5 mg, or 0.2 mg to 10 mg, with doses of 0.1 mg, 0.2 mg, 0.3 mg, 0.4 mg, 0.5 mg, 0.6 mg, 0.7 mg, 0.8 mg, 0.9 mg, 1.5 mg, 1.0 mg, 1.75 mg, 2 mg, 2.5 mg, 2.75 mg, 3 mg, 3.5 mg, 3.75 mg, 4 mg, 4.5 mg, 4.75 mg, 5 mg, 5.5 mg, 6 mg, 6.5 mg, 7 mg, 7.5 mg, 8 mg, 8.5 mg, 9 mg, 10 mg, 11 mg, 12 mg, 15 mg, 20 mg, 25 mg, and 30 mg being specific examples of doses.

Typically, dosages of gaboxadol or a pharmaceutically acceptable salt thereof are administered once or twice daily to a patient in need thereof. The methods and compositions described herein may provide reduced dosing frequency and reduced adverse events and/or increased efficacy. In embodiments, the dosage is about, e.g., 0.1-20 mg/day, or 0.2-15 mg/day, or 0.5-10 mg/day, or 0.75-5 mg/day, for example 0.2 mg/day, 0.5 mg/day, 0.75 mg/day, 1 mg/day, 1.5 mg/day, 2 mg/day, 3 mg/day, 4 mg/day, 5 mg/day, 6 mg/day, 7 mg/day, 8 mg/day, 9 mg/day, 10 mg/day, 11 mg/day, 12 mg/day, 13 mg/day, 14 mg/day, 15 mg/day, 16 mg/day, 17 mg/day, 18 mg/day, 19 mg/day, or 20 mg/day. In embodiments, gaboxadol or a pharmaceutically acceptable salt thereof, or a derivative or analogue thereof is administered at doses of 0.2 mg to 1 mg in infants or 1-20 mg in adults, once daily.

In embodiments, gaboxadol or a pharmaceutically acceptable salt thereof is administered on an as-needed basis by the patient. In embodiments, gaboxadol or a pharmaceutically acceptable salt thereof may be administered before a substance craving occurs. For example, the patient may take a dose of gaboxadol or a pharmaceutically acceptable salt thereof in anticipation of cravings, before a stressful situation occurs, or when facing another trigger for substance use. In embodiments, the patient takes a dose of gaboxadol or a pharmaceutically acceptable salt thereof after the substance craving occurs, for example during the craving, in order to reduce or eliminate the craving. In embodiments, the dose of gaboxadol or a pharmaceutically acceptable salt thereof is low enough, e.g., less than 15 mg, less than 10 mg, or less than 5 mg, that a patient can take one dose before a craving occurs, and another later the same day if he/she feels or anticipates another craving.

In embodiments, gaboxadol is provided as gaboxadol monohydrate. One skilled in the art will readily understand that the amounts of active ingredient in a pharmaceutical composition will depend on the form of gaboxadol provided. For example, pharmaceutical compositions of including 5.0, 10.0, or 15.0 mg gaboxadol correspond to 5.6, 11.3, or 16.9 mg gaboxadol monohydrate.

In embodiments, gaboxadol is crystalline, such as the crystalline hydrochloric acid salt, the crystalline hydrobromic acid salt, or the crystalline zwitter ion monohydrate. In embodiments, gaboxadol is provided as a crystalline monohydrate.

In embodiments, gaboxadol may be formulated for administration to a patient using pharmaceutically acceptable salts including acid addition salts, a zwitter ion hydrate, zwitter ion anhydrate, hydrochloride or hydrobromide salt, or in the form of the zwitter ion monohydrate. Acid addition salts, include but are not limited to, maleic, fumaric, benzoic, ascorbic, succinic, oxalic, bis-methylenesalicylic, methanesulfonic, ethane-disulfonic, acetic, propionic, tartaric, salicylic, citric, gluconic, lactic, malic, mandelic, cinnamic, citraconic, aspartic, stearic, palmitic, itaconic, glycolic, p-amino-benzoic, glutamic, benzene sulfonic or theophylline acetic acid addition salts, as well as the 8-halotheophyllines, for example 8-bromo-theophylline. In other suitable embodiments, inorganic acid addition salts, including but not limited to, hydrochloric, hydrobromic, sulfuric, sulfamic, phosphoric or nitric acid addition salts may be used.

In embodiments, gaboxadol or a pharmaceutically acceptable salt thereof is administered via a pharmaceutical composition. Pharmaceutical compositions (also referred to simply as compositions) herein encompass dosage forms. Dosage forms herein encompass unit doses. In embodiments, as discussed below, various dosage forms including conventional formulations and modified release formulations can be administered one or more times daily. Any suitable route of administration may be utilized, e.g., oral, rectal, nasal, pulmonary, vaginal, sublingual, transdermal, intravenous, intraarterial, intramuscular, intraperitoneal and subcutaneous routes. Suitable dosage forms include tablets, capsules, oral liquids, powders, aerosols, transdermal modalities such as topical liquids, patches, creams and ointments, parenteral formulations and suppositories.

Pharmaceutical compositions herein may be provided with conventional release or modified release profiles. Modified release profiles include immediate release, delayed release, and extended release profiles. In embodiments, pharmaceutical compositions with different drug release profiles may be combined to create a two phase or three-phase release profile. For example, pharmaceutical compositions may be provided with an immediate release and an extended release profile. In embodiments, pharmaceutical compositions may be provided with an extended release and delayed release profile. Such compositions may be provided as pulsatile formulations, multilayer tablets, or capsules containing tablets, beads, granules, etc. Compositions may be prepared using a pharmaceutically acceptable “carrier” composed of materials that are considered safe and effective. The “carrier” includes all components present in the pharmaceutical formulation other than the active ingredient or ingredients. The term “carrier” includes, but is not limited to, diluents, binders, lubricants, disintegrants, fillers, and coating compositions.

Conventional (or unmodified) release oral dosage forms such as tablets or capsules typically release medications into the stomach or intestines as the tablet or capsule shell dissolves. The pattern of drug release from modified release (MR) dosage forms is deliberately changed from that of a conventional dosage form to achieve a desired therapeutic objective and/or better patient compliance. Types of MR drug products include orally disintegrating dosage forms (ODDFs) which provide immediate release, extended release dosage forms, delayed release dosage forms (e.g., enteric coated), and pulsatile release dosage forms.

An ODDF is a solid dosage form containing a therapeutic agent or active ingredient which disintegrates rapidly, usually within a matter of seconds when placed upon the tongue. The disintegration time for ODDFs generally range from one or two seconds to about a minute. ODDFs are designed to disintegrate or dissolve rapidly on contact with saliva. This mode of administration can be beneficial to people who may have problems swallowing tablets whether it be from physical infirmity or psychiatric in nature. Examples of ODDFs include orally disintegrating tablets, capsules and rapidly dissolving films and wafers.

Extended release dosage forms (ERDFs) have extended release profiles and are those that allow a reduction in dosing frequency as compared to that presented by a conventional dosage form, e.g., a solution or unmodified release dosage form. ERDFs provide a sustained duration of action of a drug. Suitable formulations which provide extended release profiles are well-known in the art. For example, coated slow release beads or granules (“beads” and “granules” are used interchangeably herein) in which gaboxadol or a pharmaceutically acceptable salt thereof is applied to beads, e.g., confectioners nonpareil beads, and then coated with conventional release retarding materials such as waxes, enteric coatings and the like. In embodiments, beads can be formed in which gaboxadol or a pharmaceutically acceptable salt thereof is mixed with a material to provide a mass from which the drug leaches out. In embodiments, the beads may be engineered to provide different rates of release by varying characteristics of the coating or mass, e.g., thickness, porosity, using different materials, etc. Beads having different rates of release may be combined into a single dosage form to provide variable or continuous release. The beads can be contained in capsules or compressed into tablets.

In embodiments, modified dosage forms herein incorporate delayed release dosage forms having delayed release profiles. Delayed release dosage forms can include delayed release tablets or delayed release capsules. A delayed release tablet is a solid dosage form which releases a drug (or drugs) such as gaboxadol or a pharmaceutically acceptable salt thereof at a time other than promptly after administration. A delayed release capsule is a solid dosage form in which the drug is enclosed within either a hard or soft soluble container made from a suitable form of gelatin, and which releases a drug (or drugs) at a time other than promptly after administration. For example, enteric-coated tablets, capsules, particles and beads are well-known examples of delayed release dosage forms. Enteric coated tablets, capsules and particles and beads pass through the stomach and release the drug in the intestine. In embodiments, a delayed release tablet is a solid dosage form containing a conglomerate of medicinal particles that releases a drug (or drugs) at a time other than promptly after administration. In embodiments, the conglomerate of medicinal particles are covered with a coating which delays release of the drug. In embodiments, a delayed release capsule is a solid dosage form containing a conglomerate of medicinal particles that releases a drug (or drugs) at a time other than promptly after administration. In embodiments, the conglomerate of medicinal particles are covered with a coating which delays release of the drug.

Delayed release dosage forms are known to those skilled in the art. For example, coated delayed release beads or granules in which gaboxadol or a pharmaceutically acceptable salt thereof is applied to beads, e.g., confectioners nonpareil beads, and then coated with conventional release delaying materials such as waxes, enteric coatings and the like. In embodiments, beads can be formed in which gaboxadol or a pharmaceutically acceptable salt thereof is mixed with a material to provide a mass from which the drug leaches out. In embodiments, the beads may be engineered to provide different rates of release by varying characteristics of the coating or mass, e.g., thickness, porosity, using different materials, etc. In embodiments, enteric coated granules of gaboxadol or a pharmaceutically acceptable salt thereof can be contained in an enterically coated capsule or tablet which releases the granules in the small intestine. In embodiments, the granules have a coating which remains intact until the coated granules reach at least the ileum and thereafter provide a delayed release of the drug in the colon. Suitable enteric coating materials are well known in the art, e.g., Eudragit® coatings such methacrylic acid and methyl methacrylate polymers and others. The granules can be contained in capsules or compressed into tablets.

In embodiments, gaboxadol or a pharmaceutically acceptable salt thereof is incorporated into porous inert carriers that provide delayed release profiles. In embodiments, the porous inert carriers incorporate channels or passages from which the drug diffuses into surrounding fluids. In embodiments, gaboxadol or a pharmaceutically acceptable salt thereof is incorporated into an ion-exchange resin to provide a delayed release profile. Delayed action may result from a predetermined rate of release of the drug from the resin when the drug-resin complex contacts gastrointestinal fluids and the ionic constituents dissolved therein. In embodiments, membranes are utilized to control rate of release from drug containing reservoirs. In embodiments, liquid preparations may also be utilized to provide a delayed release profile. For example, a liquid preparation consisting of solid particles dispersed throughout a liquid phase in which the particles are not soluble. The suspension is formulated to allow at least a reduction in dosing frequency as compared to that drug presented as a conventional dosage form (e.g., as a solution or a prompt drug-releasing, conventional solid dosage form). For example, a suspension of ion-exchange resin constituents or microbeads.

In embodiments, methods for treating a substance use disorder, preventing a substance use craving, diminishing a substance use craving, and/or facilitating substance use cessation include administering to a patient in need thereof a pharmaceutical composition including about 0.05 mg to about 50 mg gaboxadol or a pharmaceutically acceptable salt thereof. In embodiments, methods for treating a substance use disorder, preventing a substance use craving, diminishing a substance use craving, and/or facilitating substance use cessation include administering to a patient in need thereof a pharmaceutical composition including about 0.1 mg to about 30 mg gaboxadol or a pharmaceutically acceptable salt thereof.

In embodiments, the pharmaceutical compositions include 0.1 mg to 25 mg, 0.1 mg to 20 mg, 0.1 mg to 15 mg, 0.5 mg to 25 mg, 0.5 mg to 20 mg, 0.5 to 15 mg, 1 mg to 25 mg, 1 mg to 20 mg, 1 mg to 15 mg, 1.5 mg to 25 mg, 1.5 mg to 20 mg, 1.5 mg to 15 mg, 2 mg to 25 mg, 2 mg to 20 mg, 2 mg to 15 mg, 2.5 mg to 25 mg, 2.5 mg to 20 mg, 2.5 mg to 15 mg, 3 mg to 25 mg, 3 mg to 20 mg, 3 mg to 15 mg gaboxadol or a pharmaceutically acceptable salt thereof.

In embodiments, the pharmaceutical compositions include 5 mg to 20 mg, 5 mg to 10 mg, 4 mg to 6 mg, 6 mg to 8 mg, 8 mg to 10 mg, 10 mg to 12 mg, 12 mg to 14 mg, 14 mg to 16 mg, 16 mg to 18 mg, or 18 mg to 20 mg gaboxadol or a pharmaceutically acceptable salt thereof.

In embodiments, the pharmaceutical compositions include 0.1 mg, 0.25 mg, 0.5 mg, 1 mg, 2.5 mg, 3 mg, 4 mg, 5 mg, 7 mg, 7.5 mg, 10 mg, 12.5 mg, 15 mg, 17.5 mg, 20 mg gaboxadol or a pharmaceutically acceptable salt thereof or amounts that are multiples of such doses. In embodiments, the pharmaceutical compositions include 2.5 mg, 5 mg, 7.5 mg, 10 mg, 15 mg, or 20 mg gaboxadol or a pharmaceutically acceptable salt thereof.

In embodiments, the total amount of gaboxadol or pharmaceutically acceptable salt thereof and/or gaboxadol administered to a subject in a 24-hour period is 1 mg to 50 mg. In embodiments, the total amount of gaboxadol or pharmaceutically acceptable salt thereof and/or gaboxadol administered to a subject in a 24-hour period is 1 mg to 20 mg. In embodiments, the total amount of gaboxadol or pharmaceutically acceptable salt thereof and/or gaboxadol administered to a subject in a 24-hour period is 5 mg, 10 mg, or 15 mg. In embodiments, the total amount of gaboxadol or a pharmaceutically acceptable salt thereof administered to a subject in a 24-hour period is 1 mg to 50 mg. In embodiments, the subject may be started at a low dose and the dosage is escalated. In this manner, it can be determined if the drug is well tolerated in the subject. Dosages can be lower for children than for adults. In embodiments, a dose of gaboxadol for children can be 0.1 mg/kg to 1 mg/kg.

In embodiments, compositions herein are suitable for parenteral administration, including, e.g., intramuscularly (i.m.), intravenously (i.v.), subcutaneously (s.c.), intraperitoneally (i.p.), or intrathecally (i.t.). The parenteral compositions herein must be sterile for administration by injection, infusion or implantation into the body and may be packaged in either single-dose or multi-dose containers.

In embodiments, liquid pharmaceutical compositions for parenteral administration to a subject including gaboxadol or a pharmaceutically acceptable salt thereof at a concentration of about 0.005 μg/ml to about 500 μg/ml are provided. In embodiments, the composition includes gaboxadol or a pharmaceutically acceptable salt thereof at a concentration of, e.g., about 0.005 μg/ml to about 250 μg/ml, about 0.005 μg/ml to about 200 μg/ml, about 0.005 μg/ml to about 150 μg/ml, about 0.005 μg/ml to about 100 μg/ml, or about 0.005 μg/ml to about 50 μg/ml.

In embodiments, compositions include gaboxadol or a pharmaceutically acceptable salt thereof at a concentration of, e.g., about 0.05 μg/ml to about 50 μg/ml, about 0.1 μg/ml to about 50 μg/ml, about 0.05 μg/ml to about 25 μg/ml, about 0.05 μg/ml to about 10 μg/ml, about 0.05 μg/ml to about 5 μg/ml, or about 0.05 μg/ml to about 1μg/ml. In embodiments, a composition includes gaboxadol or a pharmaceutically acceptable salt thereof at a concentration of, e.g., about 0.05 μg/ml to about 15 μg/ml, about 0.5 μg/ml to about 10 μg/ml, about 0.5 μg/ml to about 7 μg/ml, about 1 μg/ml to about 10 μg/ml, about 5 μg/ml to about 10 μg/ml, or about 5 μg/ml to about 15 μg/ml. In embodiments, pharmaceutical compositions for parenteral administration are formulated as a total volume of about, e.g., 10 ml, 20 ml, 25 ml, 50 ml, 100 ml, 200 ml, 250 ml, or 500 ml. In embodiments, compositions are contained in a bag, a glass vial, a plastic vial, or a bottle.

In embodiments, methods for treating a substance use disorder, preventing a substance use craving, diminishing a substance use craving, and/or facilitating substance use cessation by administering to a patient in need thereof a parenteral pharmaceutical composition including gaboxadol or a pharmaceutically acceptable salt thereof at a concentration of about 0.05 μg/ml to about 500 μg/ml are provided. In embodiments, the composition is disposed within a sealed glass container.

In embodiments, compositions for parenteral administration including about 0.05 mg to about 100 mg gaboxadol or a pharmaceutically acceptable salt thereof are provided. In embodiments, the pharmaceutical compositions include about, e.g., 0.1 mg to 25 mg, 0.1 mg to 20 mg, 0.1 mg to 15 mg, 0.5 mg to 25 mg, 0.5 mg to 20 mg, 0.5 to 15 mg, 1 mg to 25 mg, 1 mg to 20 mg, 1 mg to 15 mg, 1.5 mg to 25 mg, 1.5 mg to 20 mg, 1.5 mg to 15 mg, 2 mg to 25 mg, 2 mg to 20 mg, 2 mg to 15 mg, 2.5 mg to 25 mg, 2.5 mg to 20 mg, 2.5 mg to 15 mg, 3 mg to 25 mg, 3 mg to 20 mg, 3 mg to 15 mg gaboxadol or a pharmaceutically acceptable salt thereof.

In embodiments, the pharmaceutical compositions for parenteral administration include about, e.g., 5 mg to 20 mg, 5 mg to 10 mg, 4 mg to 6 mg, 6 mg to 8 mg, 8 mg to 10 mg, 10 mg to 12 mg, 12 mg to 14 mg, 14 mg to 16 mg, 16 mg to 18 mg, or 18 mg to 20 mg gaboxadol or a pharmaceutically acceptable salt thereof. In embodiments, the pharmaceutical compositions for parenteral administration include about, e.g., 0.1 mg, 0.25 mg, 0.5 mg, 1 mg, 2.5 mg, 3 mg, 4 mg, 5 mg, 7 mg, 7.5 mg, 10 mg, 12.5 mg, 15 mg, 17.5 mg, 20 mg gaboxadol or a pharmaceutically acceptable salt thereof or amounts that are multiples of such doses. The compositions may be contained in a bag, a glass vial, a plastic vial, or a bottle.

In embodiments, pharmaceutical compositions for parenteral administration to a subject include gaboxadol or a pharmaceutically acceptable salt thereof at a concentration of about 0.005 mg/ml to about 500 mg/ml. In embodiments, the compositions include gaboxadol or a pharmaceutically acceptable salt thereof at a concentration of, e.g., about 0.05 mg/ml to about 50 mg/ml, about 0.05 mg/ml to about 100 mg/ml, about 0.005 mg/ml to about 500 mg/ml, about 0.1 mg/ml to about 50 mg/ml, about 0.1 mg/ml to about 10 mg/ml, about 0.05 mg/ml to about 25 mg/ml, about 0.05 mg/ml to about 10 mg/ml, about 0.05 mg/ml to about 5 mg/ml, or about 0.05 mg/ml to about 1 mg/ml. In embodiments, the composition includes gaboxadol or a pharmaceutically acceptable salt thereof at a concentration of, e.g., about 0.05 mg/ml to about 15 mg/ml, about 0.5 mg/ml to about 10 mg/ml, about 0.25 mg/ml to about 5 mg/ml, about 0.5 mg/ml to about 7 mg/ml, about 1 mg/ml to about 10 mg/ml, about 5 mg/ml to about 10 mg/ml, or about 5 mg/ml to about 15 mg/ml. In embodiments, the pharmaceutical compositions for parenteral administration are formulated as a total volume of about, e.g., 10 ml, 20 ml, 25 ml, 50 ml, 100 ml, 200 ml, 250 ml, or 500 ml. In embodiments, the compositions are packaged and stored in a bag, a glass vial, a plastic vial, or a bottle.

In embodiments, pharmaceutical compositions including gaboxadol or a pharmaceutically acceptable salt thereof wherein the gaboxadol or pharmaceutically acceptable salt thereof is present at a molarity less than about 1.0 M are provided. In embodiments, gaboxadol or pharmaceutically acceptable salt thereof is present at a molarity greater than, e.g., about 0.0001 M about 0.001 M, about 0.01 M, about 0.1 M, about 0.2 M, greater than about 0.5, greater than about 1.0 M, greater than about 1.2 M, greater than about 1.5 M, greater than about 1.75 M, greater than about 2.0 M, or greater than about 2.5 M. In embodiments, gaboxadol or pharmaceutically acceptable salt thereof is present at a molarity of between, e.g., about 0.00001 M to about 0.1 M, about 0.01 to about 0.1 M, about 0.1 M to about 1.0 M, about 1.0 M to about 5.0 M, or about 5.0 M to about 10.0 M. In embodiments, gaboxadol or pharmaceutically acceptable salt thereof is present at a molarity of less than, e.g., about 0.01 M, about 0.1 M, about 1.0 M, about 5.0 M, or about 10.0 M.

In embodiments, the solubility of gaboxadol or pharmaceutically acceptable salt thereof in the composition is greater than, e.g., about 10 mg/mL, about 15 mg/mL, about 20 mg/mL, about 25 mg/mL, about 30 mg/mL, about 40 mg/mL, about 50 mg/mL, about 75 mg/mL, about 100 mg/mL, about 150 mg/mL, when measured, for example, in water at 25° C.

In embodiments, the solubility of gaboxadol or pharmaceutically acceptable salt thereof in the composition is between, e.g., about 1 mg/mL to about 50 mg/mL, about 5 mg/mL to about 50 mg/mL, about 10 mg/mL to about 50 mg/mL, about 20 mg/mL to about 50 mg/ml, from about 20 mg/mL to about 30 mg/mL or from about 10 mg/mL to about 45 mg/mL, when measured, for example, in water at 25° C.

In embodiments, a pharmaceutical composition for parenteral administration is provided wherein the pharmaceutical composition is stable for at least six months. In embodiments, the pharmaceutical compositions herein exhibit no more than about 5% decrease in gaboxadol or pharmaceutically acceptable salt thereof after, e.g., 3 months or 6 months. In embodiments, the amount of gaboxadol or pharmaceutically acceptable salt thereof degradation is no more than about, e.g., 2.5%, 1%, 0.5% or 0.1%. In embodiments, the degradation of gaboxadol or pharmaceutically acceptable salt thereof is less than about, e.g., 5%, 2.5%, 1%, 0.5%, 0.25%, 0.1%, for at least six months.

In embodiments, pharmaceutical compositions for parenteral administration wherein the pharmaceutical composition remains soluble are provided. In embodiments, pharmaceutical compositions that are stable, soluble, local site compatible and/or ready-to-use are provided. In embodiments, the pharmaceutical compositions herein are ready-to-use for direct administration to a patient in need thereof.

The parenteral compositions herein may include one or more excipients, e.g., solvents, solubility enhancers, suspending agents, buffering agents, isotonicity agents, stabilizers or antimicrobial preservatives. When used, the excipients of the parenteral compositions will not adversely affect the stability, bioavailability, safety, and/or efficacy of gaboxadol or pharmaceutically acceptable salt used in the composition. Thus, parenteral compositions are provided wherein there is no incompatibility between any of the components of the dosage form.

Thus, in embodiments, parenteral compositions of gaboxadol or a pharmaceutically acceptable salt thereof including a stabilizing amount of at least one excipient are provided. For example, excipients may be selected buffering agents, solubilizing agents, tonicity agents, antioxidants, chelating agents, antimicrobial agents, preservatives, and combinations thereof. One skilled in the art will appreciate that an excipient may have more than one function and be classified in one or more defined group.

In embodiments pharmaceutical compositions are provided including gaboxadol, or a pharmaceutically acceptable salt thereof, and an excipient wherein the excipient is present at a weight percent (w/v) of less than about, e.g., 10%, 5%, 2.5%, 1%, or 0.5% are provided. In embodiments, the excipient is present at a weight percent between about, e.g., 1.0% to 10%, 10% to 25%, 15% to 35%, 0.5% to 5%, 0.001% to 1%, 0.01% to 1%, 0.1% to 1%, or 0.5% to 1%. In embodiments, the excipient is present at a weight percent between about, e.g., 0.001% to 1%, 0.01% to 1%, 1.0% to 5%, 10% to 15%, or 1% to 15%.

In embodiments pharmaceutical compositions are provided including gaboxadol, or a pharmaceutically acceptable salt thereof, and an excipient wherein the excipient is present in a molar ratio of the excipient to gaboxadol or pharmaceutically acceptable salt of, e.g., about 0.01:1 to about 0.45:1, about 0.1:1 to about 0.15:1, about 0.01:1 to about 0.1:1, and about 0.001:1 to about 0.01:1 are provided. In embodiments, the excipient is present at a molar ratio of the excipient to gaboxadol or pharmaceutically acceptable salt is about 0.0001:1 to about 0.1:1 or about 0.001:1 to about 0.001:1.

In embodiments, pharmaceutical compositions are provided including gaboxadol, or a pharmaceutically acceptable salt thereof and an excipient wherein the excipient comprises a stabilizing amount of a buffering agent. The buffering agent may be used to maintain the pH of the pharmaceutical composition wherein the gaboxadol or pharmaceutically acceptable salt thereof remains soluble, stable, and/or physiologically compatible. For example, in embodiments, the parenteral compositions include a buffering agent wherein the composition remains stable without significant gaboxadol degradation. In embodiments, the addition of a buffer is desired for controlling the pH to enhance stability without significantly catalyzing or degrading the gaboxadol or salt thereof and/or causing pain to the patient upon infusion.

In embodiments, the buffering agent can be a citrate, phosphate, acetate, tartrate, carbonate, glutamate, lactate, succinate, bicarbonate buffer and combinations thereof. For example, sodium citrate, trisodium citrate anhydrous, trisodium citrate dihydrate, sodium citrate dehydrate, triethanolamine (TRIS), trisodium citrate pentahydrate, acetic acid, citric acid, glutamic acid, phosphoric acid, may be used as a buffering agent. In embodiments, the buffering agent may be an amino acid, alkali metal, or alkaline earth metal buffer. For example, the buffering agent may be sodium acetate or hydrogen phosphate.

In embodiments, provided herein are parenteral compositions of gaboxadol of pharmaceutically acceptable salts thereof wherein the pH of the composition is between about 4.0 to about 8.0. In embodiments, the pH of the compositions is between, e.g., about 5.0 to about 8.0, about 6.0 to about 8.0, about 6.5 to about 8.0. In embodiments, the pH of the compositions is between, e.g., about 6.5 to about 7.5, about 7.0 to about 7.8, about 7.2 to about 7.8, or about 7.3 to about 7.6. In embodiments, the pH of the aqueous solution of gaboxadol is, e.g., about 6.8, about 7.0, about 7.2, about 7.4, about 7.6, about 7.7, about 7.8, about 8.0, about 8.2, about 8.4, or about 8.6.

In embodiments, pharmaceutical compositions are provided including gaboxadol, or a pharmaceutically acceptable salt thereof and an excipient wherein the excipient includes a solubilizing agent. For example, solubilizing agents according to the disclosure herein may include, e.g., sodium hydroxide, L-lysine, L-arginine, sodium carbonate, potassium carbonate, sodium phosphate, and/or potassium phosphate. The amount of solubilizing agent in the composition will be sufficient such that the solution remains soluble at all concentrations, i.e., does not turn hazy and/or form precipitates.

In embodiments, provided herein are pharmaceutical compositions including gaboxadol, or a pharmaceutically acceptable salt thereof and an excipient wherein the excipient includes a particulate formation inhibitor. A particulate formation inhibitor refers to a compound that has the desired property of inhibiting the formation of particles in parenteral compositions. Particulate formation inhibitors of the disclosure herein include ethylenediaminetetraacetic acid (EDTA) and salts thereof, for example, ethylenediaminetetraacetic acid, calcium disodium salt (preferably as the hydrate); ethylenediaminetetraacetic acid, diammonium salt (preferably as the hydrate); ethylenediaminetetraacetic acid, dipotassium salt (preferably as the dihydrate); ethylenediaminetetraacetic acid, disodium salt (preferably as the dihydrate and, if desired, as the anhydrous form); ethylenediaminetetraacetic acid, tetrasodium salt (preferably as the hydrate); ethylenediaminetetraacetic acid, tripotassium salt (preferably as the dihydrate); ethylenediaminetetraacetic acid, trisodium salt (preferably as the hydrate) and ethylenediaminetetraacetic acid disodium salt, USP (preferably as the dihydrate). In embodiments, pharmaceutical compositions described herein have an effective amount of a particulate formation inhibitor. In embodiments the excipients may include, e.g., an amino acid, urea, alcohol, ascorbic acid, phospholipids, proteins, such as serum albumin, collagen, and gelatin; salts such as EDTA or EGTA, and sodium chloride, liposomes, polyvinylpyrollidone, sugars, such as dextran, mannitol, sorbitol, and glycerol, propylene glycol and polyethylene glycol (e.g., PEG-4000, PEG-6000), glycerol, glycine, and/or lipids.

In embodiments, provided herein are pharmaceutical compositions including gaboxadol, or a pharmaceutically acceptable salt thereof and an excipient wherein the excipient includes a solubilizing agent. For example, solubilizing agents may include, but are not limited to, acids, such as carboxylic acids, amino acids. In other examples, the solubilizing agents may be saturated carboxylic acids, unsaturated carboxylic acids, fatty acids, keto acids, aromatic carboxylic acids, dicarboxylic acids, tricarboxylic acids, a-hydroxy acids, amino acids, and combinations thereof.

In embodiments, provided herein are pharmaceutical compositions including gaboxadol or a pharmaceutically acceptable salt thereof and an excipient wherein the excipient includes a solubilizing agent such as formic acid, acetic acid, propionic acid, butyric acid, valeric acid, caproic acid, enanthic acid, caprylic acid, pelargonic acid, capric acid, lauric acid, stearic acid, acrylic acid, docosahexaenoic acid, eicosapentaenoic acid, pyruvic acid, benzoic acid, salicylic acid, aldaric acid, oxalic acid, malonic acid, malic acid, succinic acid, glutaric acid, adipic acid, citric acid, lactic acid, alanine, arginine, aspargine, aspartic acid, cysteine, glutamine, glycine, histidine, isoleucine, leucine, lysine, methionine, phenylalanine, praline, serine, threonine, tryptophan, tyrosine, valine, and combinations thereof.

In embodiments, the solubilizing agent is selected from acetic acid, salts thereof, and combinations thereof, (e.g., acetic acid/sodium acetate), citric acid, salts thereof and combinations thereof (e.g., citric acid/sodium citrate), DL arginine, L-arginine and histadine. In embodiments, the solubilizing agent is DL-arginine. In embodiments, the solubilizing agent is L-arginine. In embodiments, the solubilizing agent is acetic acid/sodium acetate. In embodiments, the solubilizing agent is citric acid/sodium citrate.

In embodiments, provided herein are pharmaceutical compositions including gaboxadol or a pharmaceutically acceptable salt thereof and an excipient wherein the excipient renders the composition isotonic. Isotonic pharmaceutical compositions herein may be achieved by adding an appropriate quantity of sodium chloride, glucose, laevulose, dextrose, mannitol, or postassium chloride, or calcium chloride, or calcium gluconoglucoheptonate, or mixtures thereof. For example, the excipients may include one or more tonicity agents, such as, e.g., sodium chloride, potassium chloride, glycerin, mannitol, and/or dextrose. Tonicity agents may be used to minimize tissue damage and irritation, reduce hemolysis of blood cells, and/or prevent electrolyte imbalance. For example, the parenteral compositions may be an aqueous solution including sodium chloride wherein the composition is isotonic. In embodiments, the isotonizing agent is sodium chloride. In embodiments, the concentration of the isotonizing agent is between about 0.01 and about 2.0 weight percent. In embodiments, the pharmaceutical compositions may comprise up to about 10% isotonizing agent. In embodiments the pharmaceutical compositions may comprise up to about, e.g., 0.25%, 0.5%, 1%, 2.5% isotonizing agent. In embodiments the amount of isotonizing agent in the pharmaceutical is between about, e.g., 0.01% to 1%, 0.1% to 1%, 0.25% to 1%, or 0.5% to 1%.

In embodiments, provided herein are pharmaceutical compositions including gaboxadol, or a pharmaceutically acceptable salt thereof and an excipient wherein the excipient includes a free radical antagonist. In embodiments, the free radical antagonist is ascorbic acid, ascorbic acid derivatives, organic compounds having at least one thiol, alkyl polyhydroxylated, and cycloalkyl polyhydroxylated compounds, and combinations thereof.

In embodiments, provided herein are pharmaceutical compositions including gaboxadol, or a pharmaceutically acceptable salt thereof and an excipient wherein the excipient includes a free radical scavenger selected from thiolyglycolic acid, thiolacetic acid, dithiothreitol, reduced glutathion, thiourea, α-thioglycerol, cystein, aceticystein, mercaptoethane sulfonic acid and combinations thereof.

In embodiments, provided herein are pharmaceutical compositions including gaboxadol, or a pharmaceutically acceptable salt thereof and an excipient wherein the excipient includes ribolflavin, dithiothreitol, sodium thiosulfate, thiourea, ascorbic acid, methylene blue, sodium metabisulfite, sodium bisulfite, propyl gallate acetylcysteine, phenol, acetone sodium bisulfate, ascorbic acid, ascorbic acid esters, butylhydroxyanisol (BHA), Butylhydroxytoluene (BHT), cysteine, nordihydroguiaretic acid (NDGA), monothioglycerol, sodium bisulfite, sodium metabisulfate, tocophenols, and/or glutathione.

In embodiments, provided herein are pharmaceutical compositions including gaboxadol, or a pharmaceutically acceptable salt thereof and an excipient wherein the excipient includes a preservative. In embodiments, the preservative is selected from benzalkonium chloride, benzethonium chloride, benzyl alcohol, chlorobutanol, chlorocresol, metacresol, Phenol, phenylmercuric nitrate, phenylmercuric acetate, methyl p-hydroxybenzoate, propyl p- hydroxybenzoate, butyl p-hydroxybenzoate, and thimerosal. In other embodiments, the preservative is selected from the group consisting of phenol, meta-cresol, benzyl alcohol, parabens (e.g., methyl, propyl, butyl), benzalkonium chloride, chlorobutanol, thimerosal, phenylmercuric salts (e.g., acetate, borate, or nitrate), and combinations thereof.

In embodiments, the compositions herein include a co-solvent. In some instances the solubility of gaboxadol may be well below the therapeutic dose and therefore a co-solvent system may be used. A co-solvent is a mixture of solvents that may be used to achieve sufficiently high solubility and may increase the stability. For example, co-solvents may be a water-miscible organic solvents, such as ethanol, propylene, glycol, Capmul PG, propylene glycol, glycerin, polyethylene glycol, sorbitol, dimethylacetamide, and/or dimethylsulfoxide (DMSO). In embodiments, the cosolvent may comprise up to about 75% of the pharmaceutical composition. In other embodiments the amount of cosolvent used include up to about, e.g., 1%, 5%, 10%, 15%, 25%, 40%, 50%, of the pharmaceutical composition.

The parenteral dosage forms may be prepared, for example, by mixing gaboxadol and one or more excipients (e.g., buffering agents, solubilizing agents, tonicity agents, antioxidants, chelating agents, antimicrobial agents and/or preservatives) in a blender under sterile conditions until a uniform blend is obtained. Pre-sterilized vials may then be filled with an appropriate amount of the sterile blend. The predetermined amount of sterile blend may then be mixed with a solvent, e.g., water, saline, about 5-10% sugar (e.g., glucose, dextrose) solution and combinations thereof prior to administration. In addition, the solution may be frozen and thawed prior to further processing.

The excipients may be used in solid or in solution form. When used in solid form, the excipients and gaboxadol may be mixed together as described above, and then solvent added prior to parenteral administration. When used in solution form, the gaboxadol may be mixed with a solution of the excipient prior to parenteral administration.

Parenteral solutions including gaboxadol herein, may be prepared by mixing the required amount of gaboxadol which may be purified prior to use in parenteral fluids such as D5W, distilled water, saline or PEG and adjusting the pH of this solution between 6.8-8. The process may be carried out at room temperature, or to increase concentration, the solution may be warmed appropriately. Other solvents such as PEG 400, 600, polypropylene glycol or other glycols can be used to enhance solubility. The resulting solutions after cooling to room temperature, may be sterilized by known means such as ultrafiltration using, e.g., 0.45 micron filter or ethylene oxide treatment or heating and may be packaged into ampules, vials or pre-filled syringes suitable for dispensing a sterile parenteral formulation.

When administered, the parenteral compositions herein provide a time of maximum plasma concentration (Tₘₐₓ) for gaboxadol in human patients of about 1 or more hours (e.g., about 1.5 or more hours). In embodiments, a Tₘₐₓ of gaboxadol in human patients ranging from between, e.g., about 1 to about 5 hours, about 1 to about 4 hours, about 1 to about 3 hours, about 1 to about 2 hours. In embodiments, a Tₘₐₓ for gaboxadol in human patients of more than about 1.5 is observed. In embodiments, a Tₘₐₓ for gaboxadol in human patients of less than about 3 hours is observed. The time of maximum plasma concentration is measured once infusion is complete.

In embodiments herein a dosage form includes from about 1 mg to about 500 mg gaboxadol, wherein parenteral administration (e.g., intramuscular, intravenous, subcutaneous, intraperitoneal, or intrathecal) of the dosage form provides an in vivo plasma profile for gaboxadol comprising a mean AUC_{0-∞} of more than about 25 ng⋅hr/ml. In embodiments, single dose administration of the dosage form provides an in vivo plasma profile for gaboxadol comprising a mean AUC_{0-∞} of more than about, e.g., 50 ng⋅hr/ml, 75 ng⋅hr/ml, 150 ng⋅hr/ml, 250 ng⋅hr/ml, 500 ng⋅hr/ml, 1000 ng⋅hr/ml, or 1500 ng⋅hr/ml.

In embodiments, the dosage form includes from about 1 mg to about 500 mg gaboxadol, wherein administration of the dosage form provides an in vivo plasma profile for gaboxadol comprising a mean Cₘₐₓ of less than about 10000 ng/ml. In embodiments, single dose administration of the compositions provide an in vivo plasma profile for gaboxadol of a mean Cₘₐₓ of less than about, e.g., 5000 ng/ml, 2500 ng/ml, 1000 ng/ml, 500 ng/ml, 250 ng/ml, or 100 ng/ml.

In embodiments, pharmaceutical compositions for parenteral administration include gaboxadol or a pharmaceutically acceptable salt thereof wherein parenteral administration exhibits a pharmacokinetic profile of a Tₘₐₓ at about 1 to about 120 minutes after administration of the parenteral composition; followed by a plasma drug concentration of at least 50% Cₘₐₓ for a duration of about 90 to about 360 minutes. In embodiments, parenteral administration of gaboxadol is followed by a plasma drug concentration of at least 50% Cₘₐₓ for a duration of, e.g., about 10 to about 60 minutes, about 15 to about 90 minutes, about 30 to about 120 minutes, about 60 to about 180 minutes, about 90 to about 180 minutes.

In embodiments, stable pharmaceutical compositions are provided in unit dosage form in a vial or ampoule suitable for parenteral administration having a therapeutically effective amount of gaboxadol or pharmaceutically acceptable salt thereof dissolved in sterile water to form a solution wherein the composition is substantially free of any excipient, organic solvent, buffer, acid, base, salt other than gaboxadol or pharmaceutically acceptable salt thereof. In embodiments, the pharmaceutical composition remains sufficiently soluble and is capable of direct administration. In embodiments, the pharmaceutical composition is capable of storage in the absence of an inert atmosphere for at least 6 months.

In embodiments, provided herein are stable pharmaceutical compositions in unit dosage form in a vial or ampoule suitable for parenteral administration having a therapeutically effective amount of gaboxadol or pharmaceutically acceptable salt thereof dissolved in sterile water to form a solution wherein the composition is free of any excipient, organic solvent, buffer, acid, base, salt other than gaboxadol or pharmaceutically acceptable salt thereof. In embodiments, the pharmaceutical composition remains sufficiently soluble and is capable of direct administration. In embodiments, the pharmaceutical composition is capable of storage in the absence of an inert atmosphere for at least 6 months.

In embodiments, stable pharmaceutical compositions suitable for parenteral administration include gaboxadol or a pharmaceutically acceptable salt thereof, in an aqueous solution having an osmolarity between 225 and 350 mOsm/kg and at a pH in the range between 7.0 and 8.0. In embodiments, the aqueous solution has an osmolarity between 270 and 310. In embodiments, the aqueous solution has a pH in the range between 7.2 and 7.8.

In embodiments, provided herein are methods for treating a substance use disorder, preventing a substance use craving, diminishing a substance use craving, and/or facilitating substance use cessation, e.g., for alcohol, caffeine, cannabis, hallucinogens, inhalants, opioids, sedatives, hypnotics, anxiolytics, stimulants, nicotine and tobacco, which include administering to a patient in need thereof a pharmaceutical composition including gaboxadol or pharmaceutically acceptable salt thereof wherein the composition provides improvement in at least one symptom of the substance use disorder. In embodiments, the methods provided may also surprisingly and unexpectedly reduce or prevent symptoms of the substance use disorder in a subject in need thereof.

In embodiments, methods described herein may reduce one or more symptoms of a substance use disorder in a subject after treatment compared to the absence of treatment (e.g., before treatment), or compared to treatment with an alternative conventional treatment.

In embodiments, provided herein are methods for treating a substance use disorder, preventing a substance use craving, diminishing a substance use craving, and/or facilitating substance use cessation wherein the patient is provided improvement of at least one symptom for more than 4 hours after administration of the pharmaceutical composition to the patient. In embodiments, the improvement of at least one symptom for more than 6 hours after administration of the pharmaceutical composition to the patient is provided in accordance with the present disclosure. In embodiments, improvement of at least one symptom for more than, e.g., 8 hours, 10 hours, 12 hours, 15 hours, 18 hours, 20 hours, or 24 hours after administration of the pharmaceutical composition to the patient is provided in accordance with the present disclosure. In embodiments, improvement in at least one symptom for at least e.g., 8 hours, 10 hours, 12 hours, 15 hours, 18 hours, 20 hours, or 24 hours after administration of the pharmaceutical composition to the patient is provided in accordance with the present disclosure. In embodiments, improvement in at least one symptom for 12 hours after administration of the pharmaceutical composition to the patient is provided in accordance with the present disclosure.

In embodiments, provided herein are methods for treating a substance use disorder, preventing a substance use craving, diminishing a substance use craving, and/or facilitating substance use cessation which include administering gaboxadol or a pharmaceutical salt thereof to a patient in need thereof wherein the composition provides improvement in next day functioning to the patient.

In embodiments, provided herein methods for treating a substance use disorder, preventing a substance use craving, diminishing a substance use craving, and/or facilitating substance use cessation which include administering gaboxadol or a pharmaceutical salt thereof to a patient in need thereof wherein the patient is relieved of one or more symptoms of the substance use disorder in about 2 weeks or less, 1 week or less, 1 day or less, or 1 hour or less (e.g. 15 minutes or less, half an hour or less), after administration. In embodiments, such methods may relieve the patient of at least one symptom of a substance use disorder for about 1 day or more, 1 week or more, or 2 weeks or more after administration. In embodiments, provided herein is a method including parenterally administering an effective amount of gaboxadol or a pharmaceutical salt thereof to a patient suffering from a substance use disorder, wherein the patient is substantially relieved of one or more symptoms of the substance use disorder earlier after the first administration of gaboxadol or a pharmaceutical salt thereof, as compared to the same patient administered a different anti-addictive compound.

In embodiments, provided herein are methods for treating a substance use disorder, preventing a substance use craving, diminishing a substance use craving, and/or facilitating substance use cessation wherein the amount of active substance, e.g., gaboxadol or pharmaceutically acceptable salt thereof, within the patient about 4 hours after administration of the pharmaceutical composition is less than about 75% of the administered dose. In embodiments, provided herein are methods wherein the amount of gaboxadol or pharmaceutically acceptable salt thereof within the patient about, e.g., 6 hours, 8 hours, 10 hours, 12 hours, 15 hours, or 20 hours after administration of the pharmaceutical composition is less than about 75%.

In embodiments, provided herein are methods for treating a substance use disorder, preventing a substance use craving, diminishing a substance use craving, and/or facilitating substance use cessation wherein the amount of active substance, e.g., gaboxadol or pharmaceutically acceptable salt thereof within the patient about 4 hours after administration of the pharmaceutical composition is less than about 80% of the administered dose. In embodiments, provided herein are methods wherein the amount of active substance, e.g., gaboxadol or pharmaceutically acceptable salt thereof, within the patient about, e.g., 6 hours, 8 hours, 10 hours, 12 hours, 15 hours, or 20 hours after administration of the pharmaceutical composition is less than about 80% of the administered dose.

In embodiments, provided herein are methods for treating a substance use disorder, preventing a substance use craving, diminishing a substance use craving, and/or facilitating substance use cessation wherein the amount of active substance, e.g., gaboxadol or pharmaceutically acceptable salt thereof, within the patient about 4 hours after administration of the pharmaceutical composition is between about 65% to about 85% of the administered dose. In embodiments, the amount of active substance, e.g., gaboxadol or pharmaceutically acceptable salt thereof, within the patient after about, e.g., 6 hours, 8 hours, 10 hours, 12 hours, 15 hours, or 20 hours after administration of the pharmaceutical composition is between about 65% to about 85% of the administered dose.

In embodiments, provided herein are methods for treating a substance use disorder, preventing a substance use craving, diminishing a substance use craving, and/or facilitating substance use cessation including administering to a patient in need thereof a pharmaceutical composition including an active agent, e.g., gaboxadol or pharmaceutically acceptable salt thereof, wherein the composition provides an in vivo plasma profile having a Cₘₐₓ less than about 500 ng/ml. In embodiments, the composition provides improvement for more than 6 hours after administration to the patient.

In embodiments, the composition provides an in vivo plasma profile having a Cₘₐₓ less than about, e.g., 450 ng/ml, 400 ng/ml 350 ng/ml, or 300 ng/ml and wherein the composition provides improvement of next day functioning of the patient. In embodiments, the composition provides an in vivo plasma profile having a Cₘₐₓ less than about, e.g., 250 ng/ml, 200 ng/ml 150 ng/ml, or 100 ng/ml and wherein the composition provides improvement of next day functioning of the patient.

In embodiments, provided herein are methods for treating a substance use disorder, preventing a substance use craving, diminishing a substance use craving, and/or facilitating substance use cessation including administering to a patient in need thereof a pharmaceutical composition wherein the composition provides a consistent in vivo plasma profile having a AUC_{0-∞} of less than about 900 ng⋅hr/ml. In embodiments, the composition provides improvement in next day functioning of the patient. In embodiments, the compositions provide an in vivo plasma profile having a AUC_{0-∞} of less than about, e.g., 850 ng⋅hr/ml, 800 ng⋅hr/ml, 750 ng⋅hr/ml, or 700 ng⋅hr/ml and wherein the composition provides improvement of next day functioning of the patient. In embodiments, the composition provides improvement in one or more symptom for more than 6 hours after administration.

In embodiments, provided herein are methods for treating a substance use disorder, preventing a substance use craving, diminishing a substance use craving, and/or facilitating substance use cessation including administering to a patient in need thereof a pharmaceutical composition including an active substance, e.g., gaboxadol or pharmaceutically acceptable salt thereof, wherein the composition provides an in vivo plasma profile having a AUC_{0-∞} of less than about, e.g., 650 ng⋅hr/ml, 600 ng⋅hr/ml, 550 ng⋅hr/ml, 500 ng⋅hr/ml, or 450 ng⋅hr/ml. In embodiments, the composition provides an in vivo plasma profile having a AUC_{0-∞} of less than about, e.g., 400 ng⋅hr/ml, 350 ng⋅hr/ml, 300 ng⋅hr/ml, 250 ng⋅hr/ml, or 200 ng⋅hr/ml. In embodiments, the composition provides an in vivo plasma profile having a AUC_{0-∞} of less than about, e.g., 150 ng⋅hr/ml, 100 ng⋅hr/ml, 75 ng⋅hr/ml, or 50 ng⋅hr/ml. In embodiments, the composition provides improvement of next day functioning of the patient after administration for more than, e.g., 4 hours, 6 hours, 8 hours, 10 hours, or 12 hours, after administration of the composition to the patient.

In embodiments, provided herein are methods for treating a substance use disorder, preventing a substance use craving, diminishing a substance use craving, and/or facilitating substance use cessation including administering to a patient in need thereof a first pharmaceutical composition including gaboxadol or a pharmaceutically acceptable salt thereof and a second pharmaceutical composition including gaboxadol or a pharmaceutically acceptable salt thereof. In some embodiments, the second pharmaceutical composition provides an in vivo plasma profile having a mean AUC_{0-∞} of at least about 20% less than the first pharmaceutical composition.

In embodiments, the first and/or the second pharmaceutical compositions are administered once, twice, or three times daily, or every other day. In embodiments, the first or the second pharmaceutical composition is provided to the patient in the evening. In embodiments, the second pharmaceutical composition includes an amount of gaboxadol that is at least one third of the amount of the gaboxadol or pharmaceutically acceptable salt thereof in the first pharmaceutical composition. In embodiments, the second pharmaceutical composition includes an amount of gaboxadol that is at least half of the amount of the amount of the gaboxadol or pharmaceutically acceptable salt thereof provided in the first pharmaceutical composition.

In embodiments, the first or the second pharmaceutical composition are provided to the patient once in the evening and once in the morning. In embodiments, the total amount of gaboxadol or pharmaceutically acceptable salt thereof administered to a subject in a 24-hour period is 1 mg to 100 mg. In embodiments, the total amount of gaboxadol or a pharmaceutically acceptable salt thereof administered to a subject in a 24-hour period is 1 mg to 75 mg. In embodiments, the total amount of active substance, e.g., gaboxadol or pharmaceutically acceptable salt thereof and/or gaboxadol, administered to a subject in a 24-hour period is less than about 75 mg, 50 mg, 25 mg, 20 mg, 10 mg, or 5 mg. In embodiments, the total amount of active substance, e.g., gaboxadol or pharmaceutically acceptable salt thereof and/or gaboxadol, administered to a subject in a 24-hour period is less than 15 mg.

In embodiments, a subject is provided with gaboxadol or pharmaceutically acceptable salt thereof in combination with an additional therapeutic agent. In embodiments, the effective amount of either or both of gaboxadol or pharmaceutically acceptable salt thereof and an additional therapeutic agent may be different when either is provided alone than when provided in combination. For example, when gaboxadol or pharmaceutically acceptable salt thereof and the additional therapeutic agent act synergistically, then a lower amount of gaboxadol or pharmaceutically acceptable salt thereof, a lower amount of the additional therapeutic agent, or lower amounts of both gaboxadol or pharmaceutically acceptable salt thereof and the additional therapeutic agent may be required to achieve the same therapeutic effect that would be provided by either gaboxadol or pharmaceutically acceptable salt thereof or the additional therapeutic agent alone. In embodiments, the same amount of gaboxadol or pharmaceutically acceptable salt thereof and the additional therapeutic agent are used to provide an enhanced therapeutic effect relative to the therapeutic effect provided by either gaboxadol or pharmaceutically acceptable salt thereof or the additional therapeutic agent alone.

Gaboxadol or pharmaceutically acceptable salt thereof may be effectively used in combination with one or more additional therapeutic agents for methods for treating a substance use disorder, preventing a substance use craving, diminishing a substance use craving, and/or facilitating substance use cessation, including addiction to one or more of the substances described herein. Accordingly, in embodiments, methods for treating a substance use disorder, preventing a substance use craving, diminishing a substance use craving, and/or facilitating substance use cessation, including administering to a subject addicted to substance gaboxadol or pharmaceutically acceptable salt thereof and one or more additional therapeutic agent(s), in which the gaboxadol or pharmaceutically acceptable salt thereof and the additional therapeutic agent(s) contribute to the effective treating of a substance use disorder, preventing a substance use craving, diminishing a substance use craving, and/or facilitating substance use cessation. In embodiments, a subject is provided with or administered gaboxadol or pharmaceutically acceptable salt thereof and one additional therapeutic agent. In embodiments, a subject is addicted to two or more substances.

Gaboxadol or pharmaceutically acceptable salt thereof and the additional therapeutic agent may be administered at the same time (i.e., concurrently), or either may be administered before the other (i.e., sequentially). In general, both gaboxadol or pharmaceutically acceptable salt thereof and the additional therapeutic agent may be present in the subject at the same time for a duration of time and at levels sufficient to provide a therapeutic benefit to the subject, i.e., in the treatment of a substance use disorder, prevention of a substance use craving, diminishing a substance use craving, facilitating substance use cessation and/or the prevention of a relapse use of a substance. Gaboxadol or pharmaceutically acceptable salt thereof and the additional therapeutic agent may be administered by the same or different routes of administration. In embodiments, gaboxadol or pharmaceutically acceptable salt thereof and the additional therapeutic agent can be co-administered using a composition containing both agents.

The additional therapeutic agent provided in combination with gaboxadol or pharmaceutically acceptable salt thereof may be any therapeutic agent that contributes to an aspect of the effective treatment or prevention of the substance use disorder. For example, the additional therapeutic agent may be a drug used to treat a use disorder or a drug used to alleviate side-effects associated with physiological withdrawal from a substance. In addition, the additional therapeutic agent may be any drug that affects brain serotonin neurotransmission, such as selective serotonin reuptake inhibitors (SSRIs), and tricyclic and tetracyclic serotonin and norepinephrine reuptake inhibitors (SNRIs), and serotonin agonists. In embodiments, the additional therapeutic agent can be an opioid antagonist, including mixed opioid partial agonist/antagonists, an antidepressant, an antiepileptic, an antiemetic, a dopaminergic agent such as a dopamine D1 receptor agonist, a corticotrophin-releasing factor-1 (CRF-1) receptor antagonist, a selective serotonin-3 (5-HT3) antagonist, a 5-HT2A/2C antagonist, or a cannabinoid-1 (CB1) receptor antagonist.

In embodiments, the substance is nicotine and gaboxadol or pharmaceutically acceptable salt thereof can be combined with one or more nicotine substitutes for the treatment of nicotine use disorder. Nicotine substitutes (also known as “nicotine replacement therapy” or “NRT”) may make it easier to abstain from tobacco by partially replacing the nicotine previously obtained from tobacco or other sources of nicotine. Nicotinic replacement therapies that may be combined with gaboxadol or pharmaceutically acceptable salt thereof include, but are not limited to transdermal nicotine patches (e.g., Habitrol®, Nicoderm CQ®, and Nicotrol®), nicotine gum (e.g., Nicorette®, nicotine lozenges (e.g., Commit®), nicotine-containing sublingual tablets (e.g., Nicorette® Microtabs), and nicotine nasal sprays or inhalers.

In embodiments, gaboxadol or pharmaceutically acceptable salt thereof may be combined with one or more nicotinic drugs. One particular class of nicotinic drugs that may be used encompasses α4-β2 nicotinic receptor partial agonists, including varenicline (Chantix®). Another therapeutic agent approved for the treatment of nicotine dependence is bupropion (Zyban®), which is an α3-β4 nicotinic receptor antagonist, and which can be combined with gaboxadol or pharmaceutically acceptable salt thereof. In embodiments, a substance, such as nicotine, and gaboxadol or pharmaceutically acceptable salt thereof are administered together using a transdermal patch delivery system.

In embodiments, the substance is alcohol and the additional therapeutic agent is disulfiram. In embodiments, the substance is an opioid and the additional therapeutic agent is an opioid antagonist, e.g., naltrexone, naloxone, or a mixed opioid partial agonist/antagonist, e.g., buprenorphine, nalorphine.

Unless defined otherwise, all technical and scientific terms used herein have the same meanings as commonly understood by one of skill in the art to which the disclosure herein belongs.

“PK” refers to the pharmacokinetic profile. Cₘₐₓ is defined as the highest plasma drug concentration estimated during an experiment (ng/ml). Tₘₐₓ is defined as the time when Cₘₐₓ is estimated (min). AUC_{0-∞} is the total area under the plasma drug concentration-time curve, from drug administration until the drug is eliminated (ng·hr/ml). The area under the curve is governed by clearance. Clearance is defined as the volume of blood or plasma that is totally cleared of its content of drug per unit time (ml/min).

“Excipient” is a substance, other than the active drug substance, e.g., gaboxadol, of a pharmaceutical composition, which has been appropriately evaluated for safety and are included in a drug delivery system to either aid the processing of the drug delivery system during its manufacture; protect; support; enhance stability, bioavailability, or patient acceptability; assist in product identification; or enhance any other attributes of the overall safety and effectiveness of the drug delivery system during storage or use.

“Stabilizer” or “stabilizing amount” refers to an amount of one or more excipients included in the compositions that provide sufficient stability but do not adversely affect the bioavailability, safety and/or efficacy of gaboxadol or pharmaceutically acceptable salt used in the composition.

“Stable” means that there is substantially no degradation of the gaboxadol or pharmaceutically acceptable salt thereof after a specified period of time, e.g., after 3 months or 6 months.

“Soluble” means that a solution of gaboxadol does not turn hazy and/or there is substantially no precipitate in the solution.

“Sufficiently soluble” means that the particle content is sufficiently low, and the material is sufficiently sterile such that it is useful for parenteral administration. For example, the number of particles in a liquid composition should be, e.g., less than 6,000 10 μm particles should be present in a volume of 10 ml solvent, preferably less than 10,000, less than 5,000, less than 3,000, less than 1,000, or less than 400 10 μm particles. In some examples, the number of particles in a liquid composition should be less than 1000, less than 600, or less than 200 25 μm particles in the 10 ml volume.

“Local site compatible” herein shall mean the composition is tolerant at the site of injection or infusion, thus minimizing side effects, such as local skin irritations or venous irritations, including inflammatory reactions at the infusion site. The parenteral compositions herein may have less side reactions than conventional products, such as skin irritation or phlebitis.

“Treating”, “treat” or “treatment” refers to alleviating or delaying the appearance of clinical symptoms of a disease, disorder or condition in a subject that may be afflicted with or predisposed to the disease, disorder or condition, but does not yet experience or display clinical or subclinical symptoms of the disease, disorder or condition. In certain embodiments, “treating”, “treat” or “treatment” may refer to preventing the appearance of clinical symptoms of a disease, disorder or condition in a subject that may be afflicted with or predisposed to the disease or condition, but does not yet experience or display clinical or subclinical symptoms of the disease, disorder or condition. “Treating”, “treat” or “treatment” also refers to inhibiting the disease, disorder or condition, e.g., arresting or reducing its development or at least one clinical or subclinical symptom thereof. “Treating”, “treat” or “treatment” further refers to relieving the disease, disorder or condition, e.g., causing regression of the disease, disorder or condition or at least one of its clinical or subclinical symptoms. The benefit to a subject to be treated may be statistically significant, mathematically significant, or at least perceptible to the subject and/or the physician. Nonetheless, prophylactic (preventive) and therapeutic (curative) treatment are two separate embodiments herein.

“Improvement” refers to the methods for treating a substance use disorder, preventing a substance use craving, diminishing a substance use craving, and/or facilitating substance use cessation relative to at least one symptom of the disorder.

“Improvement in one or more symptoms of a substance use disorder a day after administration” refers to improvement wherein the beneficial effect of at least one symptom lasts over a period of time, e.g., 6 hours, 12 hours, 24 hours etc.

“Pharmaceutically acceptable” refers to molecular entities and compositions that are “generally regarded as safe, e.g., that are physiologically tolerable and do not typically produce an allergic or similar untoward reaction, such as gastric upset and the like, when administered to a human. In embodiments, this term refers to molecular entities and compositions approved by a regulatory agency of the federal or a state government, as the GRAS list under section 204(s) and 409 of the Federal Food, Drug and Cosmetic Act, that is subject to premarket review and approval by the FDA or similar lists, the U.S. Pharmacopeia or another generally recognized pharmacopeia for use in animals, and more particularly in humans.

“Co-administered with”, “co-therapy”, “in combination with”, “a combination of”, “combined with” or “administered along with” may be used interchangeably and mean that two or more agents are administered in the course of therapy. The agents may be administered together at the same time or separately in spaced apart intervals. The agents may be administered in a single dosage form or in separate dosage forms.

“Patient in need thereof” includes individuals that have been diagnosed to have a substance use disorder, or to be at risk of developing a substance use disorder. “Patient” and “subject” are used interchangeably herein and include, but are not limited to, primates, canines, porcine, ungulates, rodents, poultry, and avian. The methods may be provided to any individual including, e.g., wherein the patient is a neonate, infant, a pediatric patient (6 months to 12 years), an adolescent patient (age 12-18 years) or an adult (over 18 years). It should be understood that infants can receive a pediatric dose.

“Purified” as used herein refers to material that has been isolated under conditions that reduce or eliminate the presence of unrelated materials, i.e., contaminants, including native materials from which the material is obtained. As used herein, the term “substantially free” is used operationally, in the context of analytical testing of the material. Preferably, purified material substantially free of contaminants is at least 95% pure; more preferably, at least 97% pure, and more preferably still at least 99% pure. Purity can be evaluated, for example, by chromatography or any other methods known in the art. In embodiments, purified means that the level of contaminants is below a level acceptable to regulatory authorities for safe administration to a human or non-human animal.

“Ready-to-use” with reference to the compositions herein shall mean the preparation in the reconstituted form, with standardized concentration and quality, prefilled in the single-use container, such as glass vials, infusion bags or syringes, ready for direct administration to the patient.

“Direct administration” with reference to the compositions herein shall mean the immediate administration, i.e., without further dilution, premixing with other substances or otherwise changing the composition or formulation of the composition. Such composition is typically directly discharged from an infusion device and administered via a vascular access port or through a central line.

“Dosage” is intended to encompass a formulation expressed in terms of μg/kg/day, μg/kg/hr, mg/kg/day or mg/kg/hr. The dosage is the amount of an ingredient administered in accordance with a particular dosage regimen. A “dose” is an amount of an agent administered to a mammal in a unit volume or mass, e.g., an absolute unit dose expressed in mg or μg of the agent. The dose depends on the concentration of the agent in the formulation, e.g., in moles per liter (M), mass per volume (m/v), or mass per mass (m/m). The two terms are closely related, as a particular dosage results from the regimen of administration of a dose or doses of the formulation. The particular meaning in any case will be apparent from context.

“About” or “approximately” as used herein means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value.

### EXAMPLES

The Examples provided herein are included solely for augmenting the disclosure herein and should not be considered to be limiting in any respect.

### Example 1

### Gaboxadol Plasma Concentration Profiles

The following Example provides the plasma concentration profiles and dose proportionality of gaboxadol monohydrate following single oral doses ranging from 2.5 to 20 mg. The absolute bioavailability of gaboxadol monohydrate capsules ranging from 2.5 to 20 mg is also assessed.

This study was composed of separate groups of 10 healthy adult subjects (at least 4 of each gender) who participated in a 6-period, double-blind, randomized, crossover study designed to access the dose proportionality and absolute bioavailabilty of 5 single oral doses of gaboxadol across the dose range of 2.5 to 20 mg. The order in which the subjects received the 5 single oral doses of gaboxadol (2.5; 5; 10; 15; and 20 mg) was randomized within Treatment Periods 1 through 5. Each subject was expected to complete all 6 treatment periods and there was a washout of at least 4 days between each treatment period.

Each oral dosing within Treatment Periods consisted of 2 capsules of test drug taken simultaneously at each scheduled dosing. The treatment designations for the orally administered study drugs were as follows: Treatment A—one 2.5 mg gaboxadol capsule and 1 matching placebo capsule; Treatment B—one 5 mg gaboxadol capsule and 1 matching placebo capsule; Treatment C—one 10 mg gaboxadol capsule and 1 matching placebo capsule; Treatment D—one 15 mg gaboxadol capsule and 1 matching placebo capsule; and Treatment E - 20 mg gaboxadol (two 10 mg gaboxadol capsules). Subjects received their study drug after an overnight fast with 240 mL of water in the morning about 8:00 AM. Water was permitted ad libitum except within 1 hour prior to and after study drug administration. No food was allowed for 4 hours post dose.

For each subject in each treatment, plasma and urine samples were collected over 16 hours post-dosing for the determination of pharmacokinetic parameters (e.g., AUC, Cₘₐₓ, Tₘₐₓ, apparent t_{1/2}, cumulative urinary excretion, renal clearance, clearance, and steady-state volume of distribution, as appropriate). AUC and Cₘₐₓ for gaboxadol were potency adjusted to facilitate comparison of pharmacokinetic data across studies. Table 1 provides the individual potency-adjusted pharmacokinetic parameters of gaboxadol following single oral doses (2.5, 5, 10, 15, and 20 mg).

TABLE 1
Pharmacokinetic parameters for gaboxadol following oral and IV administration

| | Geometric Mean (N = 10) | | | | | | |
|---|---|---|---|---|---|---|---|
| | | | 10 mg | 10 mg | | | |
| Parameter | 2.5 mg | 5 mg | Oral | I.V. | 15 mg | 20 mg | Slope (90% CI) ^{††} |
| AUC_{0-∞} | 90 | 171 | 346 | 380 | 539 | 669 | 0.98 (0.95, 1.01) |
| (ng · hr/mL) | | | | | | | |
| Cₘₐₓ (ng/mL)^{†} | 61 | 110 | 232 | 212 | 382 | 393 | 0.95 (0.88, 1.02) |
| Tₘₐₓ (hr)^{‡} | 0.5 | 0.6 | 0.5 | — | 0.5 | 0.6 | |
| Apparent t_{1/2} (hr)^{§} | 1.5 | 1.5 | 1.6 | 1.5 | 1.5 | 1.6 | |
| CL/F (mL/min) [symbol] | 461 | 488 | 476 | 438 | 469 | 499 | |
| Fₑ (%) | 43 | 45 | 53 | 53 | 50 | 53 | |
| CL_{R} (mL/min) | 196 | 222 | 250 | 208 | 234 | 265 | |

| | |
|---|---|
| F (%) (90% CI)^{#} | 92% (0.86, 0.97) |

^{†}C_{coi} (ng/mL) for 10 mg. IV.
^{‡}Median.
^{§}Harmonic Mean. [symbol]
CL (mL/min) for 10 mg IV.
^{#}Bioavailability relative to 10 mg I.V. reference based on pooled dose-adjusted (to 10 mg) oral AUC_{0-∞} values.
^{††} Dose proportionality assessment of oral treatments only.

### Example 2

### Prospective study for Use of Gaboxadol in Smoking Cessation Treatment

Adult daily smokers motivated to quit smoking will be randomized to gaboxadol or placebo tablets and will enroll in an 8-week treatment period combining medication with brief behavioral support. Participants will be randomized into 6 separate treatment groups (A-F). Inclusion criteria for randomization require that participants are between 18 and 65 years old, must be smoking ten or more cigarettes per day and wanting to quit.

Treatment group A receives 10 mg gaboxadol in the evening. Treatment group B receives 15 mg gaboxadol in the evening. Treatment group C receives 10 mg gaboxadol in the evening and 5 mg gaboxadol in the morning. Treatment group D receives 10 mg gaboxadol in the evening and 10 mg gaboxadol in the morning. Treatment group E receives 15 mg gaboxadol in the evening and 10 mg gaboxadol in the morning. Treatment group F receives placebo in the evening and the morning.

Primary outcome measures will be rates of prolonged smoking abstinence at the end of the 8-week treatment phase. Secondary outcome measures will be: 1) 7-day point prevalence and continuous abstinence at the end of the 8-week treatment period. Measures will be assessed with a combination of self-reported tobacco use and biochemical confirmation including breath Carbon Monoxide (CO) levels and cotinine levels, and 2) prolonged smoking abstinence, 7-day point prevalence and continuous abstinence at the end of the 6-month follow-up period. Additionally, cigarette craving will be assessed at baseline, weeks 1-8, and 1, 2 & 6 month follow up. A self-report measure (Tiffany Questionnaire of Smoking Urges-Brief) will be used to assess cigarette craving.

### Example 3

### Prospective study for Use of Gaboxadol in Treatment of Alcohol Dependence

The purpose of this study will be to obtain a preliminary indication of the safety and effectiveness of oral gabaoxadol (20 mg/day) in alcohol dependent patients. Inclusion criteria for randomization require that participants are between 18 and 65 years old, meet DSM-5 criteria for current alcohol dependence. Volunteers may meet criteria for other substance abuse, or dependence on other drugs (nicotine, marijuana or cocaine) as long as the dependence on marijuana or cocaine is secondary to alcohol dependence, be medically healthy on the basis of physical examination and medical history, vital signs, ECG and laboratory tests, with a negative blood pregnancy test for females, express a desire to stop drinking alcohol, does not require any psychotropic medication, and able to provide informed consent and comply with study procedures.

The study will be a 16-week study, double-blind, parallel groups, two arm comparison of gaboxadol and placebo in patients with alcohol dependence. This outpatient clinical trial includes a 2-week placebo lead-in phase followed by a 12-week treatment phase and a 2-week lead-out phase. Patients will be seen 2x/week. There will also be a 3-month follow-up visit to reassess the status of abstinence or relapse to drinking. The design of the study is as follows:

Single-blind Placebo Lead-in Phase (Week -2 and Week -1). Patients who give informed consent and provisionally meet the inclusion-exclusion criteria will enter a 2-week Single-blind Placebo Lead-in Phase. After this placebo lead-in phase, patients will be randomized to receive gaboxadol or placebo and they will be stratified by sex, age, race and level of alcohol dependence.

12 Week Treatment Phase (Weeks 1-12). At these visits, medication will be provided, standardized assessment instruments will be completed, and biochemical measures will be taken to monitor alcohol consumption as well as compliance with study medication. In one of these two weekly visits patients will receive individual, manual-guided relapse prevention therapy with a clinician and meet with a study psychiatrist.

Single-blind Lead-out Phase (Weeks 13-14). During this 2-week lead-out phase, patients on active medication will be tapered off the medication to placebo, but all other measures and assessments (including weekly psychotherapy sessions) will remain the same. All patients, regardless of whether they complete the study or are administratively removed, will be followed up and rated at Week 14. There will also be a 3-month follow-up visit to reassess the status of abstinence or relapse to drinking.

Clinic Visits. During these visits, the research nurse and a research assistant will measure breath alcohol content (BAC), urine and blood samples as scheduled for monitoring alcohol, illicit drug use, and medication compliance. Medication compliance will initially be assessed by measuring riboflavin fluorescence in urine samples. The staff will supervise the completion of self-report questionnaires. The nurse will check vital signs at each visit, weight once per week and will question patients about compliance, any missed doses, and possible side effect or other adverse events, and will complete the compliance and adverse events forms. Medication compliance will be monitored via self-report data, collateral informant data, and medication blood levels. Likewise, drinking behavior will be monitored with BAC, self-report and collateral informant data. This data will be available to the treatment team and will be used to reinforce importance of compliance. The patient will meet weekly with the psychiatrist who will assess current alcohol use and mood status, evaluate side effects and adjust dosage as needed (using the prearranged blinded dose adjustment schedule), review events occurring since the last study visit relating to the patient's functioning, and perform the CGI-Observer rating. During one of the two weekly visits, the patient will also meet with the therapist for individual relapse prevention therapy. At any point during the trial, if the treating psychiatrist determines that the primary alcohol dependence, secondary dependence on other drugs of abuse, or new drug dependence has escalated such that a more intensive intervention is required, he/she will make decisions about removal from the trial and referral to other forms of treatment.

Medication will be given on a fixed-flexible schedule, titrated to the maximum recommended dose to minimize side effects. Patients will receive enough medication to last until the next visit. For patients assigned to gaboxadol, they will be gradually titrated from 5 mg/day to the maximal dose of 10 mg b.i.d. (20 mg/day) over the first two weeks of the treatment phase. All patients will receive matching pills in the same quantity each day. In case of several missed visits, side effects or adverse events, a study nurse and a study psychiatrist will meet with the patient and a dose decrease will be arranged using the prearranged blinded dose adjustment schedule. Patients who cannot tolerate 50% of the maximal dose (i.e., 10 mg/day gaboxadol) will be discontinued from study medication.

### Example 3

### Prospective study for Use of Gaboxadol in Treatment of Opioid Withdrawal

This will be a two-part, multicenter study to evaluate the dose-response, efficacy, and safety of gaboxadol in alleviation of symptoms in subjects undergoing total and abrupt withdrawal from short-acting opioids. Any subject dependent on short-acting opioids about to undergo opioid withdrawal will be eligible. Subjects will be evaluated for their compliance with protocol inclusion/exclusion criteria during a screening period, lasting up to 7 days.

The first part of the study will use an inpatient, randomized, double-blind, and placebo-controlled design (Days 1-7) followed by a second part, an open-label continuation treatment (Days 8-14). A total of 100 subjects will be randomized to receive gaboxadol 20 mg total daily dose (5 mg 4 times daily), gaboxadol 10 mg total daily dose (5 mg twice daily), or matching placebo in a 3:3:2 ratio (225:225:150) for 7 days (i.e., during the most intense stage of withdrawal). During the second part of the study (Days 8-14), all subjects, regardless of their treatment assignment (which will remain double-blinded), who successfully meet the definition for “completer” based on Days 1-7 (i.e., receives at least one dose of study medication on Day 7 and completes the 3.5-hour post-dose SOWS-Gossop assessment on Day 7), will be eligible to receive open-label, variable dose gaboxadol treatment (as determined by the Site Investigator, but not to exceed 20 mg/day) for up to an additional 7 days in either an inpatient or outpatient setting depending on the wishes of the investigator and the subject. No subject will receive gaboxadol for more than 14 days total from the onset of abstinence. There will be no initial dose run-up and no mandated terminal dose taper.

Efficacy and safety assessments will be made daily throughout the study. Safety will be assessed by evaluation of adverse event, clinical labs, electrocardiograms, vital signs, physical exam data, and the Columbia-Suicide Severity Rating Scale. Efficacy will be evaluated daily by subject- and observer-completed scales including the Short Opiate Withdrawal Scale of Gossop (SOWS-G)(the primary outcome measure is SOWS-G score area under the curve for Days 1-7), the Clinical Opiate Withdrawal Scales (COWS), Objective Opiate Withdrawal Scale (OOWS-Handelsman), the Visual Analog Scale for Efficacy (VAS-E), and the Modified Clinical Global Impressions scales for efficacy and side effects. Efficacy will also be evaluated by study retention, completion rates, concomitant medication use, incidence of withdrawal-related Adverse Events (AEs), and subject treatment status 30 days post last dose of study medication. Qualitative urine drug screening will be done every other day to monitor for contraband (inpatient setting) or illicit (outpatient setting) drug use. Upon a subject's exit from the study, Study Discontinuation/End of Study assessments will be done.

During the study, study drug compliance will be documented in the source daily. During the first part of the study (Days 1-7), subjects will be inpatient and each dose of study medication will be administered by study site personnel and recorded in the source. Study medication will be dosed four times daily at 8 AM, 1 PM, 6 PM and 11 PM. During the second part of the study (Days 8-14), subjects who remain inpatient will be administered each dose of study medication by study site personnel and dosing will be captured in the source. Subjects who complete the second part of the study on an outpatient basis will return to the clinic daily for study assessments. During the open-label period, subjects will be dispensed 1-2 days' worth of study drug, as needed to allow for flexibility in scheduling daily visits, to get them through until they are supposed to return the following day for additional study assessments and dosing.

Those skilled in the art will recognize, or be able to ascertain using no more than routine experimentation, many equivalents to the specific embodiments described herein. Such equivalents are intended to be encompassed by the claims.

What is claimed is:

1. A method of treating a substance use disorder comprising administering to a patient in need thereof a pharmaceutical composition comprising gaboxadol or a pharmaceutically acceptable salt thereof.

2. The method of claim 1, wherein the pharmaceutical composition comprises an effective amount of gaboxadol or a pharmaceutically acceptable salt thereof.

3. The method of claim 2, wherein the amount of gaboxadol or a pharmaceutically acceptable salt thereof is effective to reduce one or more symptoms of the substance use disorder.

4. The method of claim 1, wherein the substance use disorder is selected from the group consisting of alcohol use disorder, caffeine use disorder, cannabis use disorder, hallucinogen use disorder, inhalant use disorder, opioid use disorder, sedative use disorder, stimulant use disorder, tobacco use disorder and nicotine use disorder.

5. The method of claim 1, wherein the pharmaceutical composition comprises about 0.05 mg to about 100 mg gaboxadol or a pharmaceutically acceptable salt thereof.

6. The method of claim 1, wherein the gaboxadol or a pharmaceutically acceptable salt thereof is co-administered with an additional therapeutic agent.

7. A method for preventing a substance use craving in a patient in need thereof comprising administering to the patient a pharmaceutical composition including a prophylactically effective amount of gaboxadol or a pharmaceutically acceptable salt thereof.

8. The method of claim 7, wherein the substance is selected from the group consisting of alcohol, caffeine, cannabis, hallucinogen, inhalant, opioid, sedative, stimulant, tobacco and nicotine.

9. The method of claim 7, wherein the pharmaceutical composition comprises about 0.05 mg to about 100 mg gaboxadol or a pharmaceutically acceptable salt thereof.

10. The method of claim 7, wherein the gaboxadol or a pharmaceutically acceptable salt thereof is co-administered with an additional therapeutic agent.

11. A method for diminishing a substance use craving in a patient in need thereof comprising administering to the patient a pharmaceutical composition including an effective amount of gaboxadol or a pharmaceutically acceptable salt thereof.

12. The method of claim 11, wherein the substance is selected from the group consisting of alcohol, caffeine, cannabis, hallucinogen, inhalant, opioid, sedative, stimulant, tobacco and nicotine.

13. The method of claim 11, wherein the pharmaceutical composition comprises about 0.05 mg to about 100 mg gaboxadol or a pharmaceutically acceptable salt thereof.

14. The method of claim 11, wherein the gaboxadol or a pharmaceutically acceptable salt thereof is co-administered with an additional therapeutic agent.

15. A method of facilitating smoking cessation comprising administering to a patient in need thereof a pharmaceutical composition including gaboxadol or a pharmaceutically acceptable salt thereof.

16. The method of claim 15, wherein the pharmaceutical composition comprises an effective amount of gaboxadol or a pharmaceutically acceptable salt thereof.

17. The method of claim 15, wherein the pharmaceutical composition comprises about 0.05 mg to about 100 mg gaboxadol or a pharmaceutically acceptable salt thereof.

18. The method of claim 15, wherein the gaboxadol or a pharmaceutically acceptable salt thereof is co-administered with an additional therapeutic agent.
